SEQUENCE LISTING (27 sequences)

SEQ ID NO: 1 - DNA, 25 bp, synthetic construct
  source 1..25: mol_type = other DNA
tttagagtcactttggatgtaggtc

SEQ ID NO: 2 - DNA, 20 bp, synthetic construct
  source 1..20: mol_type = other DNA
tctgtcaagcacatccctgt

SEQ ID NO: 3 - DNA, 20 bp, synthetic construct
  source 1..20: mol_type = other DNA
acatgggacttgaagcctgt

SEQ ID NO: 4 - DNA, 20 bp, synthetic construct
  source 1..20: mol_type = other DNA
agccttcacacctgcattct

SEQ ID NO: 5 - DNA, 23 bp, synthetic construct
  source 1..23: mol_type = other DNA
ccaagtcctactgtttctcactc

SEQ ID NO: 6 - DNA, 24 bp, synthetic construct
  source 1..24: mol_type = other DNA
ctaggccacagaattgaaagatct

SEQ ID NO: 7 - DNA, 25 bp, synthetic construct
  source 1..25: mol_type = other DNA
gtaggtggaaattctagcatcatcc

SEQ ID NO: 8 - DNA, 17 bp, synthetic construct
  source 1..17: mol_type = other DNA
tgcacagtcagcaggtt

SEQ ID NO: 9 - DNA, 20 bp, synthetic construct
  source 1..20: mol_type = other DNA
agtggcctcttccagaaatg

SEQ ID NO: 10 - DNA, 20 bp, synthetic construct
  source 1..20: mol_type = other DNA
tgcgactgtgtctgatttcc

SEQ ID NO: 11 - DNA, 20 bp, synthetic construct
  source 1..20: mol_type = other DNA
gcacagtgattgccacattc

SEQ ID NO: 12 - DNA, 20 bp, synthetic construct
  source 1..20: mol_type = other DNA
aggcaaattttggtgtacgg

SEQ ID NO: 13 - DNA, 22 bp, synthetic construct
  source 1..22: mol_type = other DNA
gctagtgcagcttggaagtatt

SEQ ID NO: 14 - DNA, 20 bp, synthetic construct
  source 1..20: mol_type = other DNA
gtctgcaggcattgtaggtg

SEQ ID NO: 15 - DNA, 25 bp, synthetic construct
  source 1..25: mol_type = other DNA
  modified_base 1: mod_base = OTHER; note = 5'-terminal hexachlorofluorescein (HEX) label attached to the 5'-end
  modified_base 25: mod_base = OTHER; note = 3'-terminal black hole quencher (BHQ) tag attached to the 3'-end
catatgaaagtaggcagacttccct

SEQ ID NO: 16 - DNA, 20 bp, synthetic construct
  source 1..20: mol_type = other DNA
aaagagcagcattgcctttg

SEQ ID NO: 17 - DNA, 26 bp, synthetic construct
  source 1..26: mol_type = other DNA
  modified_base 1: mod_base = OTHER; note = 5'-terminal fluorescein amidite (FAM) label attached to the 5'-end
  modified_base 26: mod_base = OTHER; note = 3'-terminal black hole quencher (BHQ) tag attached to the 3'-end
ctatacgaagttatacaatgcctgca

SEQ ID NO: 18 - DNA, 24 bp, synthetic construct
  source 1..24: mol_type = other DNA
acagtcttgtgtggaactaagtgt

SEQ ID NO: 19 - DNA, 20 bp, synthetic construct
  source 1..20: mol_type = other DNA
tatggtccctgaggcttcct

SEQ ID NO: 20 - DNA, 27 bp, synthetic construct
  source 1..27: mol_type = other DNA
  modified_base 1: mod_base = OTHER; note = 5'-terminal hexachlorofluorescein (HEX) label attached to the 5'-end
  modified_base 27: mod_base = OTHER; note = 3'-terminal black hole quencher (BHQ) tag attached to the 3'-end
tggtatctaatgtctagtggaaagaca

SEQ ID NO: 21 - DNA, 26 bp, synthetic construct
  source 1..26: mol_type = other DNA
  modified_base 1: mod_base = OTHER; note = 5'-terminal fluorescein amidite (FAM) label attached to the 5'-end
  modified_base 26: mod_base = OTHER; note = 3'-terminal black hole quencher (BHQ) tag attached to the 3'-end
tgctatacgaagttatagacagctgg

SEQ ID NO: 22 - DNA, 21 bp, synthetic construct
  source 1..21: mol_type = other DNA
tggcattgtaaggctatggag

SEQ ID NO: 23 - DNA, 22 bp, synthetic construct
  source 1..22: mol_type = other DNA
cacaggagaatgtcaggcttat

SEQ ID NO: 24 - DNA, 25 bp, synthetic construct
  source 1..25: mol_type = other DNA
  modified_base 1: mod_base = OTHER; note = 5'-terminal 5(6)-carboxyfluorescein (56-FAM) label attached to the 5'-end
  modified_base 10: mod_base = OTHER; note = Residue at position 10 is Zen double-quenched probe (ZEN)
  modified_base 25: mod_base = OTHER; note = 3'-terminal 3' Iowa Black FQ (3IABkFQ) tag attached to the 3'-end
acatgggacnttgaagcctgtgaga

SEQ ID NO: 25 - DNA, 22 bp, synthetic construct
  source 1..22: mol_type = other DNA
gttctgtagctttctccaacca

SEQ ID NO: 26 - DNA, 19 bp, synthetic construct
  source 1..19: mol_type = other DNA
caccatgcgatcactgtcc

SEQ ID NO: 27 - DNA, 30 bp, synthetic construct
  source 1..30: mol_type = other DNA
  modified_base 1: mod_base = OTHER; note = 5'-terminal 5(6)-carboxyfluorescein (56-FAM) label attached to the 5'-end
  modified_base 10: mod_base = OTHER; note = Residue at position 10 is Zen double-quenched probe (ZEN)
  modified_base 30: mod_base = OTHER; note = 3'-terminal 3' Iowa Black FQ (3IABkFQ) tag attached to the 3'-end
agaaaatggncattgtaagcctgacattct

(19) United States
(12) Patent Application Publication
(10) Pub. No.: US 2026/0060965 A1
(43) Pub. Date: Mar. 5, 2026

(54) MODULATING THE ACUTE FOREIGN BODY REACTION TO REDUCE STENOSIS

(71) Applicant: Research Institute at Nationwide Children’s Hospital, Columbus, OH (US)

(72) Inventors: Bryce A. Kerlin, Columbus, OH (US); Toshiharu Shinoka, Columbus, OH (US); Christopher K. Breuer, New Albany, OH (US); James W. Reinhardt, Columbus, OH (US); Mackenzie Elizabeth Turner, Columbus, OH (US)

(21) Appl. No.: 19/310,766

(22) Filed: Aug. 26, 2025

Related U.S. Application Data
(60) Provisional application No. 63/687,077, filed on Aug. 26, 2024.
(60) Provisional application No. 63/704,191, filed on Oct. 7, 2024.
(60) Provisional application No. 63/755,797, filed on Feb. 7, 2025.

(51) Int. Cl. A61K 31/4365; A61K 9/00; A61K 31/4178; A61P 9/10

(52) U.S. Cl. CPC A61K 31/4365; A61K 9/0053; A61K 31/4178; A61P 9/10

(57) ABSTRACT

Therapeutic agents such as Prasugrel, a thienopyridine ADP receptor inhibitor, which inhibit ADP mediated signaling through the P2Y12 receptor, alone or in combination with an angiotensin II receptor blocker (ARB) such as Losartan that specifically blocks the angiotensin II type 1 (AT1) receptors, have been shown to be effective in reducing thrombosis and stenosis of grafts, including vascular grafts and tissue engineered vascular grafts (“TEVGs”) in multiple in vivo, and that the combination of a inhibitor of the purinergic receptors P2Y12 such as Prasugrel, with losartan, an angiotensin II type 1 receptor inhibitor, more than additively reduced the incidence of TEVG stenosis. In a preferred embodiment, a multi-dosing unit for treatment to reduce stenosis delivers a 2-week course of losartan and Prasugrel.

### CROSS-REFERENCE TO RELATED APPLICATIONS

This application claims priority to U.S. Ser. No. 63/687,077 filed Aug. 26, 2024, U.S. Ser. No. 63/704,191 filed Oct. 7, 2024, and U.S. Ser. No. 63/755,797 filed Feb. 7, 2025.

### STATEMENT REGARDING FEDERALLY SPONSORED RESEARCH OR DEVELOPMENT

This invention was made with government support under grant nos. HL157491, HL098039, HL098228, HL176054, and DK124549 awarded by the National Institutes of Health. The government has certain rights in the invention.

### FIELD OF THE INVENTION

This invention is generally in the field of reducing pathological remodeling in tissue engineered vascular conduits, vascular grafts. stents, patches, and replacement heart valves.

### INCORPORATION BY REFERENCE

The Sequence Listing XML file named “NWCH_2024-017-05_US_ST26” created on Nov. 17, 2025, and having a size of 33,364 bytes, is hereby incorporated by reference.

### BACKGROUND OF THE INVENTION

Vascular restenosis is the re-narrowing of a blood vessel after it has been widened or opened, typically following a procedure like angioplasty or stent placement. It occurs as a result of the body's natural healing process, which can lead to excessive tissue growth (neointimal hyperplasia) and remodeling of the vessel wall. This narrowing can restrict blood flow and potentially lead to symptoms or complications. Restenosis is essentially a wound-healing response in the blood vessel, where the body tries to repair the damage caused by procedures like angioplasty or stent placement. The process involves cell migration and proliferation (especially smooth muscle cells), as well as remodeling of the vessel wall, which can lead to the narrowing of the lumen.

Abnormal proliferation of tissue is a common result of surgical procedures to implant stents and vascular grafts, tissue engineered grafts, and treatments such as balloon angioplasty to re-open clogged arteries. An arterial stenosis is a narrowing of the lumen that disturbs the local blood flow and precludes the adequate irrigation of perfused organs. The treatment of stenotic arterial segments relies on surgical grafting or medical minimally invasive procedures such as stenting. However, both methods often lead to intimal hyperplasia resulting from uncontrolled proliferation of vascular smooth myocytes. Whereas atheroma evolves during a time magnitude order of 10 years, post-therapeutic intimal hyperplasia develops in a period of about one month.

Currently, the only method to reduce restenosis is to use drug eluting stents (DES). FDA approved DES release drugs such as sirolimus or paclitaxel that inhibit the growth of this excess tissue (neointimal hyperplasia), which is the primary cause of restenosis. DES have dramatically reduced the restenosis rate compared to bare-metal stents, with rates ranging from 5% to 10%. However, restenosis can still occur with DES, although this may occur later than with bare-metal stents, sometimes even after the first year. Factors that can increase the risk of restenosis with DES include diabetes, longer lesions, smaller vessels, chronic kidney disease, and lesions in vein grafts.

Prosthetic materials are often used in surgical procedures to repair or replace tissues that are diseased, damaged, or congenitally absent. Implantation of prosthetic materials induces a foreign body reaction, a complex host response that consists of overlapping inflammatory and wound healing processes. The foreign body reaction is necessary for implant integration. However, undesired outcomes may arise from sustained activation of the immune response. For example, when synthetic materials are used in vascular repair (i.e., vascular grafts), they are associated with an increased risk of thromboembolic events occurring shortly after operation and poor long-term durability due to neointimal hyperplasia or dystrophic calcification. Autologous vascular conduits mitigate these risks and reduce postoperative complications, but this approach is usually not possible due to the limited availability of autologous tissue for surgical repair or replacement.

As reviewed by Soares, et al., App. Engin. Sci. 12, 100114 (2022), over the last two decades, Breuer and co-workers have established an immense body of work on tissue engineered vascular grafts (“TEVGs”), focusing specifically on in vivo function. In the late 1990s and early 2000s, Shinoka and Hibino successfully developed large diameter TEVGs based on PGA-PLA-PCL scaffolds seeded with a mixture of autologous cells in several animal models.

Promising results were quickly transitioned into human intervention with TEVGs made with autologous bone marrow-derived mononuclear cells (BM-MNCs) seeded onto a biodegradable tubular scaffold fabricated from a PGA fiber-based mesh coated with a 50:50 copolymer of polycaprolactone and PLA. The first implantation of this TEVG was reported in 2001 in a 4-year-old girl with single ventricle and pulmonary atresia reconstructing the occluded pulmonary artery (Shinoka, et al., N Engl J Med 2001; 344:532-533 (2001); and soon after, reports of the same procedure done on 22 more pediatric patients with single ventricle physiology followed in a pilot study in Japan (Matsumura, et al., J. Thoracic Cardiovascular Surgery. 129(6): 1330-1338 (2005)). Given the promising results of the Japan trial and the further refinement of the technology with large animal models, Breuer and coworkers initiated a clinical trial in the US to evaluate these TEVGs under the regulation of the US Food and Drug Administration (FDA). Unfortunately, this study was placed on hold only after 4 pediatric implantations due to high incident of early graft stenosis.

Drews, et al., Sci. Transl. Med. 12:6919 (2020) reported that computational modeling of the growth and remodeling processes of in vivo function of TEVGs during their inflammatory-mediated integration suggested that the acute stenosis observed in the US-FDA would have resolved without intervention. It was not observed in the Japanese trial because of the lack of consistent, early medical imaging. The authors demonstrated that changes in TEVG geometry, thickness, and stiffness affected patient-specific hemodynamics but remained within normal ranges despite the clinically observed graft narrowing.

TEVG stenosis can arise from either mural thrombus or vascular wall remodeling including either wall thickening or inward remodeling. Many efforts have been made to address these issues, e.g., heparin conjugation (Matsuzaki, et al., Ann. Thorac. Surg. 111, 1234-1241 (2021) or administration of Interleukin-10 (Mirhaidari et al., Adv. Healthc. Mater. 9, 2001094 (2020)) to prevent thrombosis; and treatment with angiotensin II type 1 receptor inhibitor (losartan, de Dios Ruiz-Rosado, et al., 2018 FASEB J. 32, 6822-6832), TGF-β R1 inhibitors (Duncan, et al., 2015 J. Am. Coll. Cardio. 65, 512-514; Lee, et al., 2016 FASEB J. 30, 2627-2636), or cilostazol (Tara, et al., 2015 Arterioscler. Thromb. Vasc. Biol. 35, 2003-2010), to mitigate adverse remodeling leading to stenosis.

The existing methods used clinically to prevent thrombosis of tissue engineered vascular grafts, the anticoagulant treatments heparin and warfarin, as well as the anti-platelet drug aspirin, are not effective. Aspirin is ineffective against this mechanism and has not been effective in animals that received a vascular graft. No therapies have been completely effective.

None have been completely effective. The existing methods used clinically to prevent thrombosis of tissue engineered vascular grafts, the anticoagulant treatments heparin and warfarin, as well as the anti-platelet drug aspirin, are not effective.

Bioresorbable vascular grafts offer the potential of a synthetic conduit that ultimately transforms into a neovessel capable of growth throughout the lifespan of the host patient. However, neotissue hyperplasia leading to stenosis is the primary cause of graft failure in a clinical trial, evaluating these grafts in the treatment of congenital heart disease. Aspirin, which is expected to prevent platelet aggregation on the graft, promoting smooth muscle cell proliferation causing neotissue hyperplasia, does not prevent the development of neotissue hyperplasia in a mouse inferior vena cava implantation model.

It is therefore an object of the present invention to provide a method and drug dosage forms and dosing regimens to optimize vascular neotissue formation using therapeutic agents to reduce mural thrombosis of tissue engineered grafts and subsequent stenosis.

### SUMMARY OF THE INVENTION

A method of reducing mural thrombosis or stenosis of vascular grafts, stents, valves, and blood vessels following implantation or angioplasty has been developed wherein an effective amount of an inhibitor of ADP-mediated signaling or the purinergic receptors P2Y12, P2Y1, and P2X1 on the surface of platelets is administered (both in dosage and time of administration) to reduce thrombosis or stenosis of the graft, stent, or valve following implantation or of a blood vessel following angioplasty or anastomosis in an individual in need thereof. Therapeutic agents such as Prasugrel, a thienopyridine ADP receptor inhibitor, which inhibit ADP mediated signaling through the P2Y12 receptor, alone or in combination with an angiotensin II receptor blocker (ARB) such as Losartan that specifically blocks the angiotensin II type 1 (AT1) receptors, have been shown to be effective in reducing thrombosis and stenosis of grafts, including vascular grafts and tissue engineered vascular grafts (“TEVGs”) in multiple in vivo. Studies with different anti-platelet agents have shown that the mechanism of platelet aggregation and thrombus formation is primarily mediated by ADP and the purinergic receptors on the surface of platelets P2Y12, P2Y1, and P2X1.

Although other ADP inhibitors are effective to some degree, Prasugrel and cilostazol are more potent, faster acting, and more effective than clopidogrel (PLAVIX®), an earlier generation P2Y12 inhibitor. Prasugrel significantly reduces the incidence of acute tissue engineered vascular graft thrombosis and long-term graft stenosis in a mouse model, wherein the mouse abdominal inferior vena cava is replaced with a tissue engineered vascular graft (TEVG). The results demonstrate a reduction in the incidence and severity of clinical TEVG stenosis.

Prasugrel effectively prevents TEVG stenosis/occlusion by inhibiting ADP mediated platelet signaling through the P2Y12 receptor. In a study in which unseeded TEVGs were implanted into P2Y12 KO mice, which carry a global deletion of the P2Y12 receptor that binds to ADP and potentiates platelet activation and thrombus stability, P2Y12 KO inhibited the development of stenosis, with an overall 100% patency rate (n=24, 12/sex).

Additional studies using losartan showed that the combination of a inhibitor of the purinergic receptors P2Y12 such as Prasugrel, with losartan, a U.S. Food and Drug Administration (FDA)-approved angiotensin II type 1 (AT-1) receptor inhibitor, synergistically reduced the numbers of macrophages in the implanted scaffolds and the incidence of TEVG stenosis. Losartan alone, prasugrel alone and prasugrel, losartan, and cell seeding together all significantly reduced stenosis but both prasugrel and prasugrel, losartan, and cell seeding eliminated stenosis (0% stenosis rate). Each drug inhibited stenosis by mitigating the foreign body reaction through the action of different cell type and different signaling pathways. Losartan blocks macrophage release from the spleen through blocking the Ang II receptor 1 and Prasugrel mediates its effect by acting on platelets by preventing aggregation by blocking P2Y12 receptor. Implantation of a foreign body (i.e., a TEVG scaffold, PTFE graft, etc. induces a foreign body reaction characterized by a time dependent infiltration of cells into the foreign body, beginning with platelets, then lymphocytes, followed by monocytes and macrophages. Each drug blocks this pathway at a different point through a different mechanism of action. Prasugrel block the P2Y12 pathway which prevents platelet aggregation on the surface of the foreign body; and losartan blocks angiotensin II receptor 1, which prevents release of monocytes from the spleen which serves as a reservoir for the monocytes/macrophages for the foreign body reaction. The combination of Prasugrel and losartan completely eliminates stenosis in the TEVG mouse model. A clinical trial using losartan combined with cell seeding reduced the incidence of stenosis but did not eliminate it. The potential risk to combining Prasugrel with the losartan to further reduce the incidence of stenosis was an increase in bleeding complications or excessive blockage of the foreign body reaction which could block the vascular neotissue formation. However, the data demonstrates losartan, Prasugrel, and cell seeding completely blocked stenosis without bleeding complications or adverse effects.

In a preferred embodiment, a multi-dosing unit for treatment to reduce stenosis delivers a 2-week course of losartan and Prasugrel. The dose of Prasugrel for adults weighing more than 60 kg, is an initial dose of 60 mg orally once followed by a 10 mg daily maintenance dose. For children, a loading dose of 0.08 mg/kg is followed by a maintenance dose of 0.04 mg/kg-0.12 mg/kg. As demonstrated for adults, a dose of 5-10 mg orally once daily may be titrated depending on the patient's blood pressure. For the pediatric population, a starting dose is 0.1 mg per kg once daily (up to 10 mg total) administered as a tablet or capsule.

Ideally administration should be started at least 1 day preoperatively and continued for a minimum of 1-3 days post-procedure to reduce or prevent platelet aggregation. In addition to blocking restenosis (which refers to development of narrowing after angioplasty or angioplasty and stenting), this could be used to prevent neointimal hyperplasia (narrowing of surgically placed vascular grafts).

### BRIEF DESCRIPTION OF THE DRAWINGS

FIG. 1A shows the cells involved in the foreign body reaction. FIG. 1B show the stages of the foreign body reaction over a period of 14 days following implantation of a tissue engineered vascular graft (“TEVG”) wherein stenotic neotissue forms from a remodeled thrombus, at zero, 3 days following migration of smooth muscle cells (“SMCs”) and endothelial cells (“ECs”) onto the graft and at 14 days, showing the developing mural thrombus.

FIG. 2A is the time post TEVG transplant. FIG. 2B is a graph of the resulting minimum luminal diameter (mm) for a wild type versus LYST mutant (2B) and wild type versus LYST mutant receiving a bone marrow transplant.

FIG. 3A is a chart of cell types, lysosome-related organelles (LROs), and clinical features when defective, showing how the animals were created. FIG. 3B is a graph of the minimum luminal diameter (mm) for wild type (WT) and global LYST mutant following TEVG implantation. FIG. 3C is a graph of the minimum luminal diameter (mm) for wild type (WT). LYST Ex52 Floxed mutant, neutrophil LYST mutant and macrophage LYST mutant following TEVG implantation.

FIG. 4A is a graph of the minimum luminal diameter (mm) for wild type (WT), global LYST mutant, and platelet LYST mutant mice following TEVG implantation. FIG. 4B is a graph of the volume (mm³) for wild type (WT), global LYST mutant, and platelet LYST mutant mice following TEVG implantation. FIG. 4C is a graph of the platelet count (×10³/mm³) for wild type (WT), global LYST mutant, and platelet LYST mutant mice following TEVG implantation.

FIG. 5 is a graph the minimum luminal diameter (mm) for wild type, floxed and Pf4-Cre mutants after TEVG implantation.

FIGS. 6A and 6B is a schematic of the remodeling following TEVG scaffold at zero, three (6A) and fourteen (6B) days after implantation, showing the aggregation, granule secretion and adhesion of platelets, and the interaction of the ADP and P2Y12 receptors on the platelets, as a thrombus/provisional matrix is formed and then remodels into a layer of smooth muscle cell having an endothelial cell layer thereon.

FIG. 7A is a graph of the minimum luminal diameter (mm) for wild type, P2Y12KO mice and prasugrel treated mice following TEVG implantation. FIG. 7B is the volume (mm³) for wild type, P2Y12KO mice and prasugrel treated mice following TEVG implantation, showing that the luminal diameter is significantly greater in both the knockout animals and the prasugrel-treated animals.

FIGS. 8A-8F are graphs comparing the effect of short term and long term losartan treatment on macrophage infiltration (FIG. 8A, F480+Ly6C+ macrophages; FIG. 8B, F480+Ly6C− macrophages), patency short-term losartan (FIG. 8D) and long-term losartan (FIG. 8E); histographs FIG. 8C. losartan, alone or in combination with bone marrow cell seeding. FIG. 8F is a graph of percent survival over time in weeks, with animals sacrificed at 24 and 52 weeks.

FIG. 9 are graphs showing minimum TEVG diameter over time (mm) in the prasugrel-losartan-cell seeded group (n=14) compared to prasugrel treated and wild type animals.

FIG. 10 is a graph of minimum luminal diameter (mm) over time (weeks). The starting scaffold diameter is indicated with the dotted line.

FIGS. 11A-11D is a comparison of patent (A, C on left) versus occluded (B, D on right) vessels following treatment.

### DETAILED DESCRIPTION OF THE INVENTION

### I. Definitions

Tissue engineered vascular graft (TEVG) refers to a conduit used to connect or bypass a blood vessel. The TEVG is made from a biodegradable scaffold. Upon implantation as the scaffold degrades, vascular neotissue forms ultimately creating an autologous neovessel which is structurally and functionally similar to a native blood vessel.

Patency refers to a conduit that is non-occluded and allows for free flow of blood.

Occlusion refers to a completely blocked conduit. Occlusion is typically due to thrombosis.

Stenosis refers to narrowing as a consequence of tissue remodeling that is guided by the initial formation of provisional matrix. Critical stenosis results in significant reduction in blood flow which can predispose to thrombosis and vascular occlusion.

Restenosis refers to development of narrowing after angioplasty or angioplasty and stenting.

ADP-mediated platelet activation and aggregation. Adenosine diphosphate (ADP) causes platelet activation and aggregation, which are important processes in hemostasis and thrombosis. ADP is released from damaged vessels and red blood cells, and also from platelet-dense granules when platelets are activated. ADP binds to receptors on platelets, causing them to change shape, secrete granules, and aggregate: P2Y receptors: ADP binds to P2Y receptors, which causes a transient rise in calcium and inhibits adenylate cyclase. Activation of the G pathway through P2Y leads to platelet shape changes and aggregation. αIIbβ3: ADP activates αIIbβ3, which also causes platelet aggregation. ADP-mediated platelet activation and aggregation can also cause secondary aggregation, and can contribute to acute coronary artery diseases, myocardial infarction, unstable angina, and stroke.

Small molecule, as used herein, refers to a molecule with a molecular weight of less than 1500 D or g/mol, more preferably less than 1000 Daltons.

Tissue engineered vascular grafts (TEVGs) are grafts that are used to repair, supplement or replace tissue. These usually consist of a material, preferably biodegradable, which is porous and shaped to repair or replace a defect, which is or can be seeded with cells.

Mural thromboses (i.e., partial) remodel over time to become tissue resembling the intima and media of a normal vessel. This tissue often resembles intimal hyperplasia in native vasculature. In a tissue engineered vascular graft, this tissue is often referred to as the neointima and the thickening as neointimal hyperplasia. Mural thrombosis directly leads to TEVG stenosis by reducing the cross-sectional area of the lumen.

Foreign body response and immune cell activation results from the direct and indirect interactions between plasma proteins and cells with an implanted material.

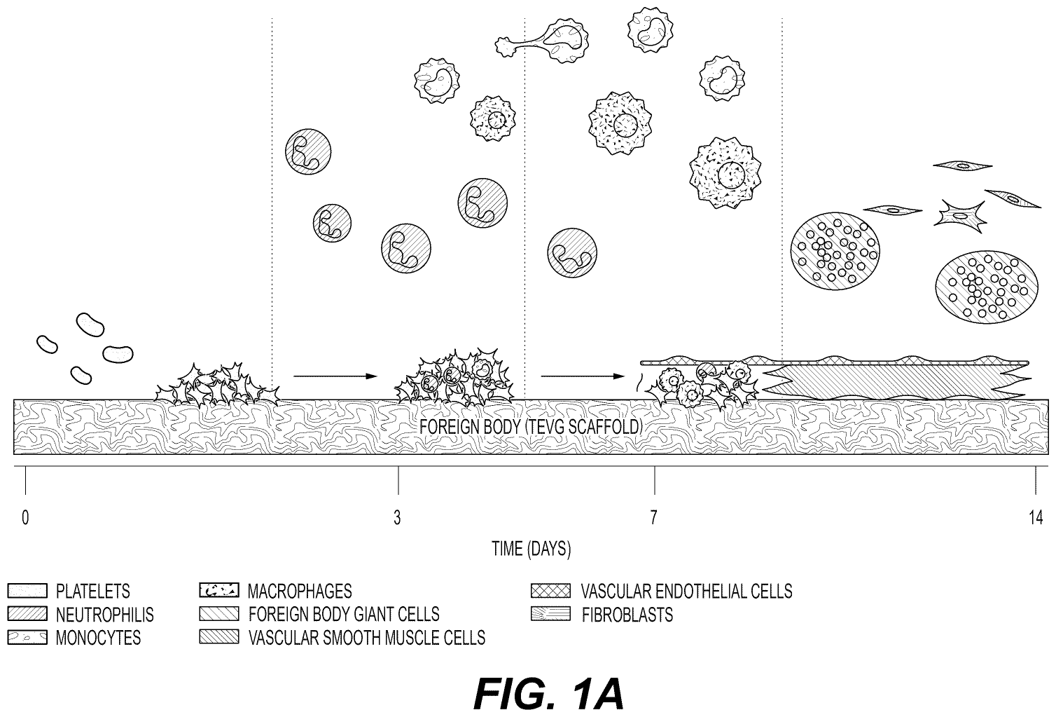

FIG. 1A shows the cells involved in the foreign body reaction. FIG. 1B show the stages of the foreign body reaction over a period of 14 days following implantation of a tissue engineered vascular graft (“TEVG”) wherein stenotic neotissue forms from a remodeled thrombus, at zero, 3 days following migration of smooth muscle cells (“SMCs”) and endothelial cells (“ECs”) onto the graft and at 14 days, showing the developing mural thrombus. This includes thrombosis, both platelet adhesion and aggregation, as well as coagulation. In addition, the foreign body reaction includes macrophage adhesion, macrophage fusion into foreign body giant cells, fibrotic encapsulation, and contractive remodeling. When platelets become activated, they secrete ADP through degranulation, which forms a positive feedback loop that promotes platelet aggregation and thrombus growth. There is infiltration by immune cells, such as macrophages and expression of MCP-1. This may be suppressed, for example, by the platelet inhibitor clopidogrel. Skewing cells toward pro-inflammatory phenotypes can promote inflammatory cytokine release. This can result in fibrosis.

Giant cell encapsulation of the polymer can lead to scaffold thickening and inward remodeling. Fibrosis and contractive remodeling create a purse string effect that can contribute to inward remodeling.

### II. Therapeutic Agents

Platelet-driven thrombosis in vivo is often described as a two-step process initiated by platelet adhesion to sites of vascular injury or foreign materials (e.g., prosthetic graft or scaffold). Sufficient activation of adherent platelets results in the self-perpetuating recruitment and accumulation of additional platelets, termed aggregation. The structure of a growing platelet-rich thrombus consists of a densely packed core region of fully activated platelets surrounded by an unstable shell of partially activated platelets. Fully activated platelets in the core begin generating txa₂ and thrombin. Production of the former is inhibited by aspirin and the latter is the primary factor for platelet activation and stabilization of aggregated platelets in the core. In addition, activated platelets in the core release platelet activating factors such as ADP and ATP and ligands such as fibrinogen as well as increase the surface expression of receptors involved in aggregation (e.g., gpiib/iiia). As a weak agonist, ADP should not on its own trigger full platelet activation and granule release prematurely before aggregation. In the shell region, ADP is primarily responsible for recruitment and retention of platelets through binding to the p2y12 receptor on the surface of circulating platelets and ADP signaling is the major determinant of overall thrombus size. In addition to p2y12 receptor signaling, ADP signaling through the p2y1 receptor and ATP signaling through the p2x1 receptor may contribute to platelet aggregation and activation.

Limited thrombosis may be beneficial since it stops and the resulting thrombus can act as a provisional matrix allowing for cell migration and provides growth factors and cytokines that stimulate angiogenesis and remodeling. Thrombosis becomes problematic when platelet aggregation is excessive and uncontrolled.

Early studies performed in c57bl/6j (wild-type, wt) mice concluded that the mechanism of stenosis was progressive narrowing, consistent with stenosis clinically. However, progressive narrowing was characterized using serial ultrasound with measurement of the TEVG luminal diameter, a practice since shown to be unreliable. An alternative explanation for stenosis revealed dense, platelet-rich mural thrombi of variable thickness formed on all scaffolds within one day after implantation, sometimes resulting in thrombotic occlusion. Evidence of ongoing platelet adhesion to the luminal surface of mural thrombi at later time points was uncommon. Pairing this with the occasional observations of thrombotic occlusion at the time of scaffold implantation indicates thrombosis is most active within minutes to hours after scaffold implantation. Thrombi were infiltrated by cells and remodeled into collagen-rich neotissue within two weeks with loss of histological evidence of platelets, fibrin, or red blood cells. Rapid thrombus remodeling explains why thrombosis-mediated occlusion was not recognized during initial studies using the mouse model as TEVGs were rarely explanted at early time points when thrombus components might still have been present. A scaffold explanted at day 10 showed that occlusive thrombi may even recanalize within this time, providing a plausible alternative to hyperplasia for the presentation of severe stenosis sometimes observed in this model.

Platelet-driven thrombosis in vivo is often described as a two-step process initiated by platelet adhesion to sites of vascular injury or foreign materials (e.g., prosthetic graft or scaffold). Sufficient activation of adherent platelets results in the self-perpetuating recruitment and accumulation of additional platelets, termed aggregation. The structure of a growing platelet-rich thrombus consists of a densely packed core region of fully activated platelets surrounded by an unstable shell of partially activated platelets. Fully activated platelets in the core begin generating txa₂ and thrombin. production of the former is inhibited by the aspirin and the latter is the primary factor for platelet activation and stabilization of aggregated platelets in the core. In addition, activated platelets in the core release platelet activating factors (e.g., ADP and ATP) and ligands (e.g., fibrinogen) as well as increase the surface expression of receptors involved in aggregation (e.g., gpiib/iiia). As a weak agonist, ADP should not on its own trigger full platelet activation and granule release prematurely before aggregation. In the shell region, ADP is primarily responsible for recruitment and retention of platelets through binding to the p2y12 receptor on the surface of circulating platelets and ADP signaling is the major determinant of overall thrombus size. In addition to p2y12 receptor signaling, ADP signaling through the p2y1 receptor and ATP signaling through the p2x1 receptor may contribute to platelet aggregation and activation.

Limited thrombosis is beneficial since it stops hemorrhaging though the porous scaffold. The thrombus also acts as a provisional matrix allowing for cell migration and provides growth factors and cytokines that stimulate angiogenesis and remodeling. Thrombosis becomes problematic when platelet aggregation is excessive and uncontrolled. The p2y12 receptor for ADP can be irreversibly inhibited by a class of drugs called thienopyridines.

### A. Inhibitors of ADP-Mediated Platelet Activation and Aggregation

P2Y12 is a G-protein-coupled receptor that is activated upon ADP binding. Blocking P2Y12 has been used to prevent antiplatelet aggregation in cardiovascular disease patients. P2Y12 is functionally expressed not only in platelets and the microglia but also in other cells of the immune system, such as in monocytes, dendritic cells, and T lymphocytes.

Adenosine-diphosphate (ADP) receptor antagonists, also known as P2Y12 receptor inhibitors, are drugs that can inhibit ADP-mediated platelet activation.

The following are representative compounds that inhibit ADP-mediated platelet activation.

### Prasugrel

Prasugrel, a thienopyridine derivative, C₂₀H₂₀FNO₃S, mw. 373.4, is a platelet activation and aggregation inhibitor structurally and pharmacologically related to clopidogrel and ticlopidine. Prasugrel is a third-generation thienopyridine that is more potent, faster acting, and lasts longer after discontinuation of treatment than clopidogrel. Prasugrel is a prodrug that requires enzymatic transformation in the liver to its active metabolite, R-138727. R-138727 irreversibly binds to P2Y12 type ADP receptors on platelets thus preventing activation of the GPIIb/IIIa receptor complex. As a result, inhibition of ADP-mediated platelet activation and aggregation occurs.

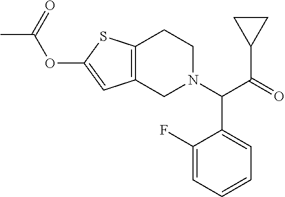 [SMILES: CC(=O)Oc1cc2c(s1)CCN(C(C(=O)C1CC1)c1ccccc1F)C2]

When platelets become activated at the TEVG surface, they secrete ADP through degranulation, which forms a positive feedback loop that promotes further aggregation and thrombus growth. Prasugrel inhibits stenosis by interfering with this sustained platelet aggregation, which reduces the size of the developing thrombus.

Due to decreased aggregation, there are fewer platelets degranulating and therefore decreased local concentrations of platelet-derived inflammatory mediators, chemokines, and growth factors. Therefore, P2Y12 inhibition may limit further cell infiltration as well as reduce the immunomodulatory effects platelets have on other cells. P2Y12 inhibition decreases the formation of monocyte platelet aggregates and dampened the platelet-mediated monocyte activation.

P2Y12 signaling in monocytes and macrophages seems to be pro-healing or pro-fibrotic. While other cells do express P2Y12, the primary signaling occurs through platelets, and P2Y12 stimulation increases the platelet surface expression of other receptors involved in platelet function. P2Y12 inhibition may indirectly or directly impact the following processes: immune cell infiltration, immune cell activation, and fibrosis.

As demonstrated in the examples, Prasugrel effectively inhibited stenosis while preserving vascular neotissue formation at 14 days, demonstrating that inhibiting ADP-mediated platelet signaling prevents aberrant remodeling without hindering vascular neotissue formation.

Prasugrel is an irreversible inhibitor, and with the exception of juvenile platelets, platelets do not have mRNA. Therefore, platelets cannot synthesize new P2Y12 receptors, and prasugrel treatment leads to sustained inhibition of P2Y12-mediated activation for the duration of a platelet's lifespan, which approximately is three to five days in mice. Prasugrel also inhibits P2Y12 receptors on megakaryocytes in the bone marrow, which are then inherited by newly created platelets. Following prasugrel cessation, these newly formed platelets demonstrate impaired aggregation in response to ADP and collagen for at least two days, with function largely restored by day 5. Therefore, despite the introduction of new platelets into circulation, prasugrel-mediated inhibition is expected to remain high throughout the critical acute period (i.e., <3 days) during which occlusive thrombi form in the mouse model. As uninhibited platelets begin to replace prasugrel-inhibited platelets, collective platelet reactivity to ADP progressively increases and is expected to return to baseline within five days to one week in humans, with a more rapid recovery anticipated in mice. However, uninhibited platelets entering circulation after the critical window for occlusive thrombosis encounter a different environment than the pro-thrombotic surface present shortly after implantation. Even after one day, the platelet-rich thrombus at the luminal surface appears contracted in size with reduced porosity. Based on morphology, these platelets are expected to have exhausted their granules, and the compact structure would likely limit the release of platelet activating factors (e.g., ADP and TXA₂), as well as the accumulation of concentrations sufficient to meaningfully recruit additional platelets. By day 3, platelet-rich mural thrombus covered by a thin layer of fibrin with loosely entrapped RBCs is observed. The sparse presence of platelets in this fibrin layer suggests that it has minimal platelet-activating potential. Within two weeks, endothelial cells are present in large numbers on the luminal surface, further protecting against platelet adhesion and activation, with full endothelialization observed by four weeks. Thus, despite the eventual restoration of platelet responsiveness to ADP, the post-acute environment is not conducive to rapid thrombus growth or the formation of occlusive thrombi in the mouse model.

Although PRASUGREL is the preferred inhibitor of ADP platelet mediated activation as described herein, others that inhibit ADP mediated signaling through the P2Y1 and P2X1 receptors, as well as the P2Y12 receptor, may be useful. The initial response to ADP is a change in the shape of the platelet whereby disc shaped cells convert into a spherical form from which pseudopodia emerge. This change, which is mediated by the P2Y1 receptor, involves Ca²⁺ influx, intracellular Ca²⁺ mobilization and actin polymerization. Interaction of ADP with the P2Y12 receptor results in inhibition of adenylate cyclase, which is accompanied by platelet aggregation.

Other Inhibitors of Platelet Aggregation Binding to P2Y1 and/or P2Xi Receptors:
GLS-409 (Gremmel, et al. Arterioscler Thromb Vasc Biol 2016 Jan. 7; 36(3): 501-509 doi: 10.1161/ATVBAHA.115.306885)
MRS2179 (Baurand, et al. Eur J Pharmacol. 2001; 412:213-21. doi: 10.1016/s0014-2999(01) 00733-6)
2-Chloro N (6)-methyl-(N)-methanocarba-2′-deoxyadenosine-3′,5′-bisphosphate (Boyer et al. Br J Pharmacol. 2002; 135:2004-10. doi: 10.1038/sj.bjp.0704673)
MRS2500 [2-iodo-N6-methyl-(N)-methanocarba-2′-deoxyadenosine-3′,5′-bisphosphate] (Hechler B, et al. J Pharmacol Exp Ther. 2006; 316:556-63. doi: 10.1124/jpet.105.094037) MRS2500 [2-iodo-N6-methyl-(N)-methanocarba-2′-deoxyadenosine-3′,5′-bisphosphate] is a potent, selective, and stable antagonist of the platelet P2Y1 receptor with strong antithrombotic activity in mice.
Acyl derivatives of coenzyme A which inhibit platelet function via antagonism at P2Y1 and P2Y12 receptors (Manolopoulos et al. Platelets. 2008; 19:134-45. doi: 10.1080/09537100701708498).
P2Y1 receptor antagonists (Pfefferkorn et al. Bioorg Med Chem Lett. 2008; 18:3338-43. doi: 10.1016/j.bmcl.2008.04.028).
Benzofuran-substituted urea derivatives (Thalji et al. Bioorg Med Chem Lett. 2010; 20:4104-7. doi: 10.1016/j.bmcl.2010.05.072. Kunapuli et al. Curr Pharm Des. 2003; 9:2303-16. doi: 10.2174/1381612033453947.

### Inhibitors of Platelet Aggregation Having Other Mechanisms:

### Clopidogrel

Clopidogrel, C₁₆H₁₆ClNO₂S, mw 321.82 g mol⁻¹, a thienopyridine used to reduce the risk of ischemic stroke, myocardial infarction, and vascular death.

Clopidogrel, sold under the brand name PLAVIX® and ISCOVER® among others, is used to reduce the risk of heart disease and stroke in those at high risk. It is taken by mouth. Its effect starts about two hours after intake and lasts for five days. Clopidogrel is considered safe and well-tolerated.

### Ticlopidine

Ticlopidine is a thienopyridine, 5-(2-Chlorobenzyl)-4,5,6,7-tetrahydrothieno[3,2-c]pyridine, a member of monochlorobenzenes.

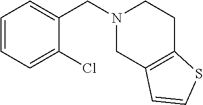 [SMILES: Clc1ccccc1CN1CCc2sccc2C1]

It is a fibrin modulating drug, a hematologic agent, an anticoagulant, a platelet aggregation inhibitor and a P2Y12 receptor antagonist. It is a prodrug that is metabolized to an active form, which blocks the ADP receptor that is involved in GPIIb/Illa receptor activation leading to platelet aggregation.

Ticlopidine is marketed under the brand name TICLID® and is indicated for patients who cannot take aspirin or in whom aspirin has not worked to prevent a thrombotic stroke.

### Ticagrelor

Ticagrelor, C₂₃H₂₈F₂N₆O₄S, has a mw of 522 g mole.

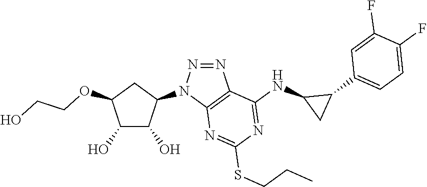 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

Unlike clopidogrel, ticagrelor is not a prodrug. It is marketed by Astra Zeneca as BRILINTA® in the US and BRILIQUE® or POSSIA® in the EU. Recommended for STEMI patients, ticagrelor can reduce ischemic outcomes without significantly increasing the risk of bleeding.

These drugs are irreversible and can inhibit platelet function for the life of the platelet, which is 7-10 days. They can also impair platelet aggregation and fibrinogen-mediated platelet cross-linking, and may be effective in preventing cardiovascular disease.

Adenosine is an inhibitor of platelet aggregation that acts via A2A and A2B receptors on platelets.

Caffeic acid, a water-soluble component of Radix Salvia miltiorrhiza, can inhibit ADP-induced platelet aggregation in vitro and thrombus formation in vivo, and GLS-409, which can inhibit ADP-stimulated platelet aggregation in a dose-dependent manner.

Inhibitors of the p2y12 Receptor for ADP

Thienopyridines can inhibit platelet activation by purinergic receptors.

Apyrase, also known as cd39 or ecto-nucleoside triphosphate diphosphohydrolase 1 (e-ntpdase1), is expressed non-exclusively in mice and humans. Apyrase inhibits platelet activation by dephosphorylating ADP and ATP to AMP. AMP may be further dephosphorylated by CD73 to adenosine, which is an inhibitor of platelet activation, though in vivo studies suggest that increased CD39 activity has a greater influence on platelet inhibition than the accumulation of adenosine. Apyrase is not as effective as a platelet inhibitor such as prasugrel.

### Angiotensin II Receptor Blocker (ARB) Inhibitors

Losartan, chemically known as 2-butyl-4-chloro-1-[p-(o-1H-tetrazol-5-ylphenyl)benzyl]imidazole-5-methanol, is an angiotensin II receptor blocker (ARB). It is commonly marketed as the potassium salt, losartan potassium. The molecule features a biphenyl group with a tetrazole ring, which acts as a bioisostere for a carboxylic acid group, and an imidazole ring, along with other substituents.

The tetrazole ring is crucial for its ability to block angiotensin II receptors. Losartan can directly interact with and bind to the thromboxane A2 (TXA2) receptor on platelets. This binding is believed to be a key mechanism by which losartan inhibits platelet aggregation and activation. Losartan is primarily known as an angiotensin II receptor blocker (ARB), specifically targeting the AT1 receptor. While this action is primarily linked to blood pressure regulation, the activation of the AT1 receptor in platelets can also lead to increased platelet activation. By blocking the AT1 receptor, losartan indirectly reduces platelet activation.

Losartan is used to reduce TEVG stenosis by inhibiting the recruitment of monocytes from the spleen. Studies have indicated an important contribution of spleen-derived monocytes to the pathology observed in experimental models of atherosclerosis, stroke, and myocardial infarction. The angiotensin II/AT-1 signaling pathway has been shown to participate in the rapid deployment of Ly6C+hi monocytes from the spleen.

The AT1 receptor for angiotensin II (Ang II) that is inhibited by losartan is not only found on vascular smooth muscle cells, but also on platelets. Intravenous infusion of angiotensin II increases markers of platelet secretion and the platelet surface expression of P-selectin. Studies have shown that physiological levels of plasma Ang II have a potentiating effect on platelet function. Inhibition of AT1R using losartan reduces thrombosis in vivo and platelet aggregation in vitro. Ang II signaling through the AT1R dose-dependently increases the sensitivity of platelets to activation by increasing cytosolic levels of Ca²⁺. Ang II has a biphasic effect on platelet aggregation, increasing the sensitivity of platelets to aggregatory stimuli up to a threshold, beyond which Ang II has platelet inhibitory effects. However, this threshold appears to be in the supraphysiologic range. In a variety of pathological conditions (e.g., hypertension, CKD), Ang II levels are elevated. For example, in single-ventricle patients Ang II levels following TCPC (42 pmol/L), BDG (40 pmol/L), or TCPC and BDG (42 pmol/L) increased nearly 4-fold compared to healthy controls (11 pmol/L). Increased levels of Ang II increase platelet sensitivity to activation and aggregation and platelet inhibition by losartan is expected to have a greater effect.

Losartan has been described to inhibit aggregation of platelets by collagen by blocking clustering of GPVI without blocking binding of collagen. In flow whole-blood, losartan suppressed the formation of multi-layered platelet thrombi at arteriolar shear rates at concentrations that that hardly affect collagen-induced platelet aggregation in platelet rich plasma.

### Angiotensin-Converting Enzyme (“ACE”) Inhibitors

ACE inhibitors block the angiotensin-converting enzyme (ACE), which is part of the renin-angiotensin-aldosterone system (RAAS). Common ACE inhibitors include lisinopril, enalapril, and ramipril. Similar to direct AT1 inhibition, ACE inhibition, upstream of Angi II production, also reduces intimal hyperplasia and platelet aggregation. Chronic ACE inhibition has been shown to reduce intimal hyperplasia associated with vein graft stenosis and after balloon-catheter injury. Ang II, the ligand for the AT1R, stimulates hypertrophy in cultured smooth muscle cells, and induces production of the proto-oncogenes c-fos and c-myc. Fosinoprel also can significantly reduce platelet aggregation under conditions in which plasma ADP levels are increased.

ACE inhibitors like Lisinopril and ARBs like Losartan work through different mechanisms. ACE inhibitors block the formation of angiotensin II, while ARBs like losartan block the action of angiotensin II by preventing it from binding to its receptors. Both, however, can thereby reduce platelet activation.

Macrophage cell density (cells/area) within the vascular graft is correlated with the incidence of stenosis. A greater density of macrophages is found in stenotic compared to patent vascular grafts. Losartan can be used to reduce vascular graft stenosis by inhibiting the recruitment of monocytes from the spleen. The angiotensin II/AT-1 signaling pathway has been shown to participate in the rapid deployment of Ly6C+hi monocytes from the spleen.

The AT1 receptor for angiotensin II (Ang II) that is inhibited by losartan is not only found on vascular smooth muscle cells, but also on platelets. Intravenous infusion of angiotensin II increases markers of platelet secretion and the platelet surface expression of P-selectin. Physiological levels of plasma Ang II have a potentiating effect on platelet function. Inhibition of AT1R using losartan reduces thrombosis in vivo and platelet aggregation in vitro. Ang II signaling through the AT1R dose-dependently increases the sensitivity of platelets to activation by increasing cytosolic levels of Ca²⁺. Ang II has a biphasic effect on platelet aggregation, increasing the sensitivity of platelets to aggregatory stimuli up to a threshold, beyond which Ang II has platelet inhibitory effects. However, this threshold appears to be in the supraphysiologic range. In a variety of pathological conditions (e.g., hypertension, CKD), Ang II levels are elevated. Increased levels of Ang II increase platelet sensitivity to activation and aggregation and platelet inhibition by losartan is expected to have a greater effect.

Losartan can inhibit aggregation of platelets by collagen by blocking clustering of GPVI without blocking binding of collagen. In flowing whole-blood, losartan suppressed the formation of multi-layered platelet thrombi at arteriolar shear rates at concentrations that that hardly affect collagen-induced platelet aggregation in platelet rich plasma.

Angiotensin-converting enzyme (ACE) inhibition, upstream of Angi II production, also reduces intimal hyperplasia and platelet aggregation. Chronic ACE inhibition has been shown to reduce intimal hyperplasia associated with vein graft stenosis and after balloon-catheter injury. Ang II, the ligand for the AT1R, stimulates hypertrophy in cultured smooth muscle cells, and in duces production of the proto-oncogenes c-fos and c-myc. Fosinoprel (ACE inhibitor) can significantly reduce platelet aggregation under conditions in which plasma ADP levels are increased. At 10 nM or below, Ang II promotes aggregability and PKC phosphorylation in human platelets through the AT1 receptor, which can be inhibited by AT1 receptor antagonists. Isolated platelets aggregate in response to low (pM), but not high (nM), concentrations of angiotensin II. The platelet aggregation response to 10 pM angiotensin II was dependent on AT1-receptors.

### III. Methods of Use

The inhibitor(s) of ADP-mediated platelet activation preferably is administered prior to surgery to implant a biodegradable vascular device, such as a polylactide-glycolide (“PLGA”) tissue engineered used as a fontan conduit during the surgical repair of congenital heart defects in the pediatric population, a vascular stent, a patch, anastomosis, or before angioplasty.

As described in the examples, prasugrel is the preferred therapeutic agent, alone or in combination with losartan. t has been discovered that prasugrel and losartan act together in more than an additive manner to reduce stenosis or restenosis.

In the preferred embodiment an inhibitor of ADP-mediated platelet activations such as prasugrel is administered up to a few hours prior to surgery, preferably two hours before the surgery.

For adults weighing more than 60 kg, the general guidance for prasugrel is to give an initial dose of 60 mg orally once followed by a 10 mg maintenance dose. For children, one study used a loading dose of 0.08 mg/kg followed by a maintenance dose of 0.04 mg/kg-0.12 mg/kg (absolute maximum).

For adults, the general guidance is to give a dose of 50 mg of Losartan orally once. This may be titrated depending on the patient's response. For the pediatric population, the usual recommended starting dose is 0.7 mg per kg (12-16 kg general weight at Fontan operation) once daily (up to 50 mg total) administered as a tablet. Thus, the expected range would be around 8 mg-12 mg daily.

Losartan's active metabolite has a half-life of 6-9 hours. Prasugrel's active metabolite has a half-life of approximately 7 hours. Importantly, prasugrel is an irreversible inhibitor and will hinder platelet function for the life span of the affected platelet (i.e., 7-10 days). The drugs should be given repeatedly to ensure lasting effects on the foreign body response to the TEVG.

Prasugrel should be given prior to surgery until the patient shows responsiveness. After surgery, the patient should begin losartan treatment. The combination of these drugs should be given until historical peak stenosis is observed (i.e., six months post-implantation). The foreign body response is active long after the TEVG is implanted. Although the thrombus transforms into collagen-rich neotissue, this neotissue is still capable of activating platelets and stimulating inflammation. It is not expected that the TEVG surface will be fully protect against thrombosis and stenosis until the endothelium is confluent and functional. Since stenosis is spontaneously reversible and begins to subside after six months, a six-month course of treatment should be effective at preventing stenosis.

Losartan's active metabolite has a half-life of 6-9 hours. Prasugrel's active metabolite has a half-life of ˜7 hours. Importantly, prasugrel is an irreversible inhibitor and will hinder platelet function for the life span of the affected platelet (i.e., 7-10 days). The drugs should be given repeatedly to ensure lasting effects on the foreign body response to the TEVG.

### IV. Dosage Units

The platelet aggregation inhibitors, preferably Prasugrel and losartan, can be provided in a dosage unit package, with dosages separated by time of administration, to insure the correct dose is administered from prior to the surgery to the conclusion of the treatment. These are typically administered orally. In the preferred embodiment, a dosage unit package is provided which contains a two week course of Prasugrel and Losartan. Prasugrel and Losartan are more effective in combination than either alone.

Prasugrel should be given prior to implant until the patient shows responsiveness. After TEVG implantation, the patient should begin losartan treatment. The combination of these drugs should be given until historical peak stenosis is observed (i.e., six months post-implantation). The foreign body response is active long after the TEVG is implanted. Although the thrombus transforms into collagen-rich neotissue, this neotissue is still capable of activating platelets and stimulating inflammation. The TEVG surface is not fully protected against thrombosis and stenosis until the endothelium is confluent and functional. Since stenosis is spontaneously reversible and begins to subside after six months, a six-month course of treatment should be effective at preventing stenosis. Losartan's active metabolite has a half-life of 6-9 hours. Prasugrel's active metabolite has a half-life of about 7 hours. Importantly, prasugrel is an irreversible inhibitor and will hinder platelet function for the life span of the affected platelet (i.e., 7-10 days). The drugs should be given repeatedly to ensure lasting effects on the foreign body response to the TEVG.

In summary, recommended clinical dosing of prasugrel:

For adults weighing 60 kilograms (kg) or more: 60 milligrams (mg) taken as a single loading dose, and then 10 mg once a day. For adults weighing less than 60 kg, 60 mg taken as a single loading dose, and then 5 mg once a day.

For children, use and dose is primarily measured by assessing platelet reactivity, which reflects the drug's effectiveness in inhibiting platelet aggregation. Several devices and assays are used for this purpose:

VerifyNow P2Y12 Assay: This is a point-of-care (POC) device that is widely used to assess the P2Y12-mediated platelet aggregation. It reports results in P2Y12 Reaction Units (PRU), with lower PRU values indicating greater platelet inhibition. The Verify Now system measures platelet-induced aggregation as an increase in light transmittance. This assay is sensitive and specific for P2Y12 inhibition and is considered a well-standardized and reproducible test. The assay uses ADP and prostaglandin E1 to measure the effects of prasugrel on the P2Y12 receptor.

Light Transmission Aggregometry (LTA): LTA is considered the gold standard for measuring platelet aggregation in vitro. However, it is a more complex and time-consuming method and is typically used in research settings or when other standardized assays are unavailable. With LTA, ADP is used to stimulate platelet-rich plasma, and the inhibition of platelet aggregation is calculated as the percentage decrease in aggregation compared to baseline.

### Controlled/Extended-Release Formulations

Although in the preferred embodiment the compounds such as prasugrel and losartan are given orally, they may be formulated to provide extended release. The platelet activation inhibitor can be incorporated into the biodegradable scaffold, graft, valve or stent to be implanted, where it is eluted from the scaffold or graft.

Alternatively, the platelet aggregation inhibitor can be administered in an modified release formulation that releases an effective dosage over a period of 24, 48, 72 or 96 hours, or an equivalent thereof. A modified release dosage form is one for which the drug release characteristics of time course and/or location are chosen to accomplish therapeutic or convenience objectives not offered by conventional dosage forms such as solutions, ointments, or promptly dissolving dosage forms. Delayed release, extended release, and pulsatile release dosage forms and their combinations are types of modified release dosage forms.

A delayed release dosage form is one that releases a drug (or drugs) at a time other than promptly after administration. An extended-release dosage form is one that allows at least a two-fold reduction in dosing frequency as compared to that drug presented as a conventional dosage form (e.g. as a solution or prompt drug-releasing, conventional solid dosage form). A pulsatile release dosage form is one that mimics a multiple dosing profile without repeated administration and allows at least a twofold reduction in dosing frequency as compared to the drug presented as a conventional dosage form (e.g. as a solution or prompt drug-releasing, conventional solid dosage form).

These formulations are well known in the pharmaceutical industry. In some embodiments drug-resin complexes are coated with a pH sensitive polymer which is insoluble in the acid environment of the stomach, and soluble in the more basic environment of the GI tract. The outer coating prevents drug release in the stomach. Preventing drug release in the stomach has the advantage of reducing side effects associated with irritation of the gastric mucosa. Avoiding release within the stomach can be achieved using enteric coatings known in the art. The enteric coated formulation remains intact or substantially intact in the stomach, however, once the formulation reaches the small intestines, the enteric coating dissolves and exposes either drug containing ion-exchange resin particles or drug-containing ion-exchange resin particles coated with extended-release coating. The enteric coated particles can be prepared as described in references such as “Pharmaceutical dosage form tablets”, eds. Liberman et. al. (New York, Marcel Dekker, Inc., 1989), “Remington—The science and practice of pharmacy”. 20th ed., Lippincott Williams˜Wilkins, Baltimore, MD, 2000, and “Pharmaceutical dosage forms and drug delivery systems”, 6th Edition, Ansel et. al., (Media, PA: Williams and Wilkins, 1995).

Examples of suitable coating materials include, but are not limited to, cellulose polymers, such as cellulose acetate phthalate, hydroxypropyl cellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Rohm Pharma). Examples of suitable coating materials include, but are not limited to, cellulose polymers, such as cellulose acetate phthalate, hydroxypropyl cellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit˜(Rohm Pharma). Additionally, the coating material may contain conventional carriers such as plasticizers, pigments, colorants, glidants, stabilisation agents, and surfactants.

In general, any coating procedure which provides a contiguous coating on each particle of drug-resin complex without significant agglomeration of particles may be used. Coating procedures known in the pharmaceutical art including, but not limited to, fluid bed coating processes and microencapsulation may be used to obtain appropriate coatings. The coating materials may be any of a large number of natural or synthetic film-formers used singly, in admixture with each other, and in admixture with plasticizers (for example, Durkex 500 vegetable oil), pigments and other substances to alter the characteristics of the coating. In general, the major components of the coating should be insoluble in, and permeable to, water. However, it might be desirable to incorporate a water-soluble substance, such as methyl cellulose, to alter the permeability of the coating. The coating materials may be applied as a suspension in an aqueous fluid or as a solution in organic solvents. The water-permeable diffusion barrier may consist of ethyl cellulose, methyl cellulose and mixtures thereof. As used herein, the term water-permeable is used to indicate that the fluids of the alimentary canal will permeate or penetrate the coating film with or without dissolving the film or parts of the film. Depending on the permeability or solubility of the chosen coating (polymer or polymer mixture) a lighter or heavier application thereof is required to obtain the desired release rate. The water-permeable diffusion barrier may also consist of water insoluble synthetic polymers sold under the trade name Eudragit (Rohm Pharma), such as Eudragit RS, Eudragit RL, Eudragit NE and mixtures thereof. other examples of such coating materials can be found in the Handbook of Pharmaceutical Excipients, Ed. By A. Wade and P. J. Weller, (1994).

These dosage units and methods of use are made clearer by reference to the following non-limiting examples.

### Example 1: Determination of which Cells Initiate Stenosis and Specific Molecular Targets

There is an urgent need for improved biomaterials that perform more like autologous tissue. To this end, tissue engineered vascular grafts (TEVGs) that form autologous neovessels have been developed. The TEVG scaffolds incite a foreign body response consisting of overlapping inflammatory and wound healing processes, guiding neovessel formation as the scaffold degrades. However, this response can lead to aberrant remodeling, where excessive neotissue growth leads to clinically significant vessel narrowing that may necessitate patients to undergo additional interventions.

A murine model was used to investigate the molecular and cellular mechanisms governing the early foreign body response to TEVGs. Following TEVG implantation in mice, platelets adhere to the scaffold, forming a layer of densely packed platelets, or develop into an occlusive, platelet-rich thrombus. Over two weeks, the resultant provisional matrix remodels into collagen-rich vascular neotissue including a neointima and neomedia through the recruitment and activity of other cells.

The early foreign body response to TEVG implantation was investigated to understand how innate immune interactions contribute to subsequent tissue remodeling. Following TEVG implantation, platelets adhere to the scaffold, forming a layer of densely packed platelets, or when signaling occurs in excess, the platelets develop into an occlusive, platelet-rich thrombus.

Over two weeks, the resultant provisional matrix, initially composed of platelets and blood proteins, remodels into collagen-rich neotissue through the recruitment and activity of other immune cells which are critical for ensuring proper neovessel formation. In a large diameter vascular graft, such as those used in the clinical trial, excessive tissue remodeling manifests as narrowing; however, in the small animal model, overgrowth of neotissue could lead to complete occlusion early in the remodeling process. This has been observed by day 3 in the mouse model.

In mice, it was found that the SCID/Beige double mutation reduced stenosis, whereas the SCID mutation alone was insufficient to produce this effect. This finding indicated that the beige mutation, which affects the lysosomal trafficking regulator gene (Lyst), significantly decreased the incidence of TEVG stenosis, even in the absence of cell seeding. The Lyst gene encodes a protein involved in innate immune cell function, which is integral to the early foreign body response, including inflammation and wound healing.

The role of Lyst-mediated innate immune function in the foreign body reaction to TEVG scaffolds using cell-specific Lyst mutant mice was used to determine the type of cells and receptors that initiate stenosis. Seeded and unseeded TEVGs were implanted into LYST Exon52 deletion mice (global LYST mutant) and C57BL/6J (WT) mice to study the role of LYST mutations in aberrant TEVG remodeling,

### Materials and Methods

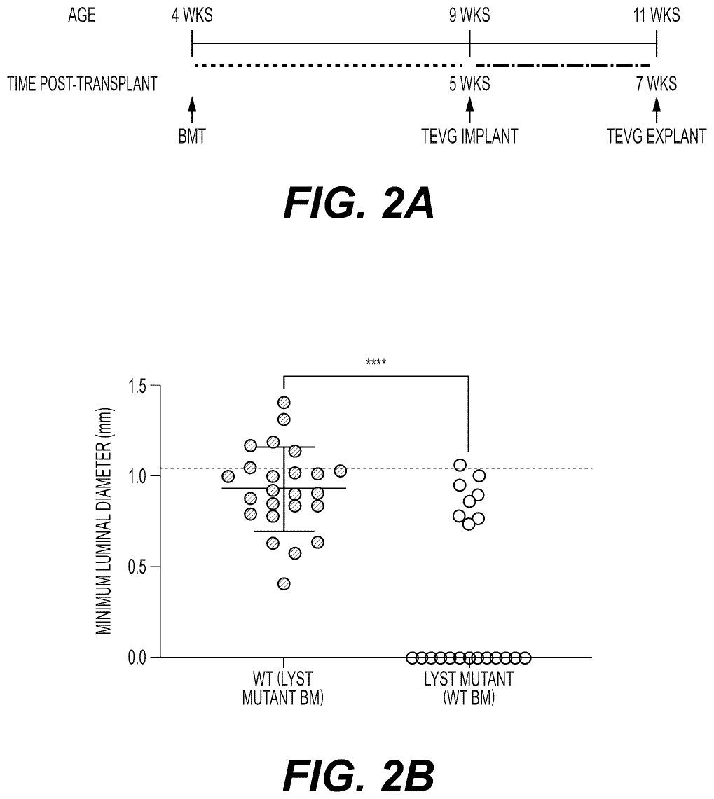

Cell-specific LYST mutants were created to evaluate key contributors to LYST-mediated stenosis and neotissue formation as shown in FIG. 3A. Studies then focused on platelets and other early infiltrating cell types known to play important roles in neotissue formation.

TEVGs were implanted into macrophage, neutrophil, and megakaryocyte/platelet specific LYST mutant mice to determine whether cell specific LYST mutations could improve TEVG performance.

All animal experiments were done in accordance with the NIH (MD, USA) institutional guidelines for the use and care of laboratory animals. The Institutional Animal Care and Use Committee (IACUC) at Nationwide Children's Hospital approved the experimental procedures and experiments involving the use of animals. All mice were housed in a specific pathogen-free facility and kept in a temperature-controlled room set to a light and dark cycle of 12 h each. Mice were provided with ad libitum access to standard chow and water.

### Scaffold Fabrication

TEVG scaffolds (3.0 mm in length, average of 1.05 mm inner diameter) were created using nonwoven polyglycolic acid (PGA) felt, which was sealed with a 50:50 copolymer solution of ε-caprolactone and ι-lactic acid (PCL/LA). These scaffolds were produced by Gunze Ltd. (Japan), sterilized with ethylene oxide gas, and stored at −20° C. until ready for implantation.

### Animal Preparation

In partnership with The Jackson Laboratory (Bar Harbor, ME), CRISPR/Cas9 was used to produce a LYST exon 52 floxed mouse line (Lyst Ex52^{fl/fl}) on a C57BL/6J background. These mice were crossed with Sox2-Cre mice to generate progeny with widespread deletion of LYST exon 52, which harbors the 3 base-pair deletion linked to the Beige (Bg) mouse strain. Subsequent breeding removed the Cre element, yielding the C57BL/6J-LYSTem52J/J strain (stock #407964) characterized by a germline deletion of LYST exon 52 (global LYST mutant). These Lyst Ex52^{fl/fl} mice were bred to iPf4-Cre, S100A8-Cre, and Lyz2-Cre transgenic mice (The Jackson Laboratory) to generate megakaryocyte/platelet, neutrophil, and macrophage specific LYST mutant mice, respectively. The megakaryocyte/platelet specific mutant is simplified to platelet specific LYST mutant.

TABLE 1
LYST MUTATIONS

| Strain | Mutation | Mutation Target Cells | JAX strain # |
|---|---|---|---|
| C57Bl6/J | Wild-type (WT) | N/A | 000664 |
| Global LYST mutant | Germline Lyst Exon 52 deletion (Sox2-Cre) | All cells | NA |
| B6.129P2-Lyz2^{tm1(cre)Ifo}/J | Macrophage specific LYST Exon 52 | Macrophages | 004781 |
| | deletion (Lyz2-Cre/LysMcre) | | |
| | “Macrophage specific LYST mutant” | | |
| B6.Cg-Tg(S100A8- | Neutrophil specific LYST Exon 52 | Neutrophils | 021614 |
| cre, -EGFP)1Ilw/J | deletion (S100A8-Cre/MRP8-Cre- | | |
| | ires/GFP) “Neutrophil specific LYST | | |
| | mutant” | | |
| C57BL/6-Tg(Pf4- | Megakaryocyte/platelet specific LYST Exon | Megakaryocytes and | 008535 |
| icre)Q3Rsko/J | 52 deletion (Pf4-Cre/Cxcl4-iCre) “Platelet | platelets | |
| | specific LYST mutant” | | |
| Lyst Ex52^{fl/fl} | LoxP flanked LYST Exon 52 “Floxed” | All cells | NA |
| C57BL/6J-Lyst^{bg-J}/J | Beige (Bg) or Lyst^{bg} | All cells | 000629 |

### LYST Genotype Confirmation and Gene Expression Quantification

The LYST genotypes were monitored and determined using PCR followed by electrophoresis, utilizing custom primers designed and manufactured by Integrated DNA Technologies (Coralville, IA). Two PCR primer sets assessed WT and mutant LYST expression, with real-time PCR confirming the presence of LoxP sites flanking LYST exon 52. See Table 2.

TABLE 2
Lyst Mutant Mice Genotypes

| SEQ ID NO | PCR Assay | ID | Sequence | Note |
|---|---|---|---|---|
| SEQ ID NO: 1 | General LYST Ex52 | 50978 | 5′ TTT AGA GTC ACT TTG GAT GTA GGT C 3′ | MUT Forward |
| | KO | | | |
| SEQ ID NO: 2 | General LYST Ex52 | 48628 | 5′ TCT GTC AAG CAC ATC CCT GT 3′ | MUT Reverse |
| | KO | | | |
| SEQ ID NO: 3 | General LYST Ex52 | 50976 | 5′ ACA TGG GAC TTG AAG CCT GT 3′ | WT Forward |
| | KO | | | |
| SEQ ID NO: 4 | General LYST Ex52 | 50977 | 5′ AGC CTT CAC ACC TGC ATT CT 3′ | WT Reverse |
| | KO | | | |
| SEQ ID NO: 5 | Pf4-Cre | 19003 | 5′ CCA AGT CCT ACT GTT TCT CAC TC 3′ | Transgene Forward |
| SEQ ID NO: 6 | Pf4-Cre | oIMR7338 | 5′ CTA GGC CAC AGA ATT GAA AGA TCT 3′ | Internal Positive |
| | | | | Control Forward |
| SEQ ID NO: 7 | Pf4-Cre | oIMR7339 | 5′ GTA GGT GGA AAT TCT AGC ATC ATC C 3′ | Internal Positive |
| | | | | Control Reverse |
| SEQ ID NO: 8 | Pf4-Cre | oIMR8669 | 5′ TGC ACA GTC AGC AGG TT 3′ | Transgene Reverse |
| SEQ ID NO: 9 | S100A8-Cre | 31704 | AGT GGC CTC TTC CAG AAA TG | Internal Positive |
| | | | | Control Forward |
| SEQ ID NO: 10 | S100A8-Cre | 31705 | TGC GAC TGT GTC TGA TTT CC | Internal Positive |
| | | | | Control Reverse |
| SEQ ID NO: 11 | S100A8-Cre | 59132 | GCA CAG TGA TTG CCA CAT TC | Transgene Forward |
| SEQ ID NO: 12 | S100A8-Cre | oIMR9074 | AGG CAA ATT TTG GTG TAC GG | Transgene Reverse |
| SEQ ID NO | qPCR Assay | ID | Sequence | Note |
| SEQ ID NO: 13 | LYST Ex52 Flox 5′ | 44328 | 5′ GCT AGT GCA GCT TGG AAG TAT T 3′ | 5′ Common Forward |
| SEQ ID NO: 14 | LYST Ex52 Flox 5′ | 44329 | 5′ GTC TGC AGG CAT TGT AGG TG 3′ | 5′ WT Reverse |
| SEQ ID NO: 15 | LYST Ex52 Flox 5′ | 44330 | 5′ HEX/CAT ATG AAA GTA GGC AGA CTT CCC T/BHQ3′ | 5′ HEX probe |
| SEQ ID NO: 16 | LYST Ex52 Flox 5′ | 44342 | 5′ AAA GAG CAG CAT TGC CTT TG 3′ | 5′ MUT Reverse |
| SEQ ID NO: 17 | LYST Ex52 Flox 5′ | 44343 | 5′ FAM/CTA TAC GAA GTT ATA CAA TGC CTG CA/BHQ3′ | 5′FAM probe |
| SEQ ID NO: 18 | LYST Ex52 Flox 3′ | 44344 | 5′ ACA GTC TTG TGT GGA ACT AAG TGT 3′ | 3′ Forward |
| SEQ ID NO: 19 | LYST Ex52 Flox 3′ | 44345 | 5′ TAT GGT CCC TGA GGC TTC CT 3′ | 3′ Reverse |
| SEQ ID NO: 20 | LYST Ex52 Flox 3′ | 44346 | 5′ HEX/TGG TAT CTA ATG TCT AGT GGA AAG ACA/BHQ3′ | 3′ HEX probe |
| SEQ ID NO: 21 | LYST Ex52 Flox 3′ | 44347 | 5′ FAM TGC TAT ACG AAG TTA TAG ACA GCT GG/BHQ3′ | 3′ FAM |
| | Lyst Exon 51-52-53 | 477720992 | Parent product | |
| SEQ ID NO: 22 | | 477720993 | 5′ TGGCATTGTAAGGCTATGGAG 3′ | LYST WT Forward |
| SEQ ID NO: 23 | | 477720994 | 5′ CACAGGAGAATGTCAGGCTTAT 3′ | LYST WT Reverse |
| SEQ ID NO: 24 | | 477720995 | /56-FAM/ACATGGGAC/ZEN/TTGAAGCCTGTGAGA/3IABkFQ/ | LYST WT FAM probe |
| | Lyst Exon 51-53 | 477720996 | Parent product | |
| | (52KO) | | | |
| SEQ ID NO: 25 | | 477720997 | 5′ GTTCTGTAGCTTTCTCCAACCA 3′ | LYST MUT Forward |
| SEQ ID NO: 26 | | 477720998 | 5′ CACCATGCGATCACTGTCC 3′ | LYST MUT Reverse |
| SEQ ID NO: 27 | | 477720999 | /56-FAM/AGAAAATGG/ZEN/CATTGTAAGCCTGACATTCT/3IABkFQ/ | LYST MUT FAM |
| | | | | probe |

TABLE 3
PATENCY OF IMPLANTS IN LYST MUTANTS AND PRASUGREL TREATED ANIMALS

| | | | | | Occluded | | | |
|---|---|---|---|---|---|---|---|---|
| | | Patent Min. | | | Min | | | |
| | # of | Diameter | | | Diameter | | Total | Patency |
| | Patents | (mm) | STDEV | # Occluded | (mm) | STDEV | Implanted | % |
| WT | 6 | 0.72 | 0.08 | 18 | 0 | 0 | 24 | 25.0% |
| Lyst Ex52 | 9 | 0.80 | 0.11 | 19 | 0 | 0 | 28 | 32.1% |
| Floxed | | | | | | | | |
| Macrophage | | | | | | | | |
| Specific | | | | | | | | |
| Lyst Mutant | 10 | 0.81 | 0.18 | 19 | 0 | 0 | 29 | 34.5% |
| Neutrophil | | | | | | | | |
| Specific | | | | | | | | |
| Lyst Mutant | 10 | 0.80 | 0.24 | 13 | 0 | 0 | 23 | 43.5% |
| Global Lyst | 24 | 0.98 | 0.21 | 2 | 0 | 0 | 26 | 92.3% |
| Mutant | | | | | | | | |
| Platelet | 29 | 1.00 | 0.09 | 1 | 0 | 0 | 30 | 96.7% |
| Specific | | | | | | | | |
| Lyst Mutant | | | | | | | | |
| Prasugrel | 14 | 1.00 | 0.12 | 0 | NA | NA | 14 | 100% |
| P2Y12 | 24 | 1.01 | 0.09 | 0 | NA | NA | 24 | 100% |

Cell-specific LYST expression was evaluated in target populations.

Platelets were isolated from mouse blood using established methods, and white blood cells (“WBCs”) were separated using HISTOPAQUE 1083 (Sigma-Aldrich, Burlington, MA, United States). Both cell types were lysed and homogenized with QIAshredder Spin® columns (Qiagen), and RNA was extracted with the RNeasy Mini Kit® (Qiagen, Venlo, Netherlands). cDNA synthesis was performed using the High-Capacity cDNA Reverse Transcription Kit (Applied Biosystems, Waltham, MA, United States). Custom qPCR assays compared LYST mutant and WT expression, using approximately 10 ng of platelet cDNA and 40 ng of WBC cDNA per reaction in duplicate on the STEPONEPLUS™ Real-Time PCR System (Applied Biosystems). Cycle threshold (Ct) values were normalized to ActB (Mm02619580_g1) and B2m (Mm00437762_m1) reference genes, and LYST expression was assessed using the 2^{-ΔΔCT} method.

LYST Ex52 deletion in neutrophils and macrophages was confirmed in the respective mutant models using magnetic cell sorting. Bone marrow cells from neutrophil LYST mutant mice were harvested and isolated via magnetic cell separation using the Neutrophil Isolation Kit (Miltenyi Biotec, Bergisch Gladbach, Germany). Macrophage mutant cells were obtained from peritoneal washes and sorted using the Macrophage Isolation Kit (Miltenyi Biotec, Bergisch Gladbach, Germany). Cell-specific LYST Ex52 deletion was verified using PCR.

A reciprocal bone marrow transplant (BMT) model was used to evaluate whether the cell type responsible for initiating LYST-mediated TEVG stenosis originates in the bone marrow. Recipient mice (WT or global LYST mutant; n=24/each) were irradiated prior to injection of donor bone marrow cells from either LYST mutant or WT mice. The high dose of radiation allowed for nearly complete reconstitution of the recipient immune system with donor cells. Five weeks after BMT, TEVGs were implanted into the transplanted mice and were explanted 14 days later. Diameters were obtained using in vivo Micro-CT scans.

### Platelet-Rich Plasma (PRP) Collection

Whole blood was collected from the abdominal inferior vena cava (“IVC”) of anesthetized mice (0.6-1.0 mL) into a syringe containing ˜0.02 mL of 0.03% citrate. Blood was centrifuged at 200×g at room temperature to separate the platelet-rich plasma, which was then suspended in Tyrode's buffer for further experimentation. Platelet count and purity were determined using a hematology analyzer.

### Platelet Count

Whole blood was collected from the submandibular vein of mice anesthetized with 1.5% v/v inhaled isoflurane into a citrated tube. To survey platelet count in the WT, global LYST mutant, platelet specific LYST mutant mice, platelet numbers in the whole blood were counted using a fully automated ABX Micros 60 hematology analyzer (Horiba Ltd., Kyoto, Japan).

### Platelet Aggregometry

Adenosine 5′-diphosphate (ADP), adenosine 5′-triphosphate (ATP), thrombin, and collagen were obtained from CHRONO-LOG® Corporation (CHRONO-LOG® Corporation, Havertown, PA). Platelet rich plasma was collected. Undisturbed platelets were added to a glass cuvette with a metal stirrer and placed in the lumi-aggregometer. CHRONO-LUME® was added for quantifying ATP release, which was measured by comparing the luminescence values from each test to a previously tested ATP standard (2 nM). Two minutes later, agonist (thrombin 1 U/mL, ADP 5 mg/mL, or collagen 5 mg/mL) was introduced. Over the next six minutes, platelet aggregation was measured using light transmission and luminescence was measured as a surrogate for granule release.

### Platelet Flow Cytometry

Platelet rich plasma was collected from WT, global LYST mutant, and platelet LYST mutant mice. For each strain, 10⁶ platelets were placed in 25 μL Tyrode's buffer for three conditions: unstained, unstimulated and stained, and collagen-stimulated and stained. The stained conditions were treated with a mixture of PE-labeled antibody (emfret #M023-2) that binds to the high affinity conformation of mouse integrin αIIβIII and FITC-labeled antibody (emfret #M130-1) that reacts with mouse P-selectin (CD62P). Platelets were gated using FSC/SSC characteristics.

### Bone Marrow Transplant

Equal numbers of male and female WT mice (n=24/group) and LYST mutant mice (n=24/group) were irradiated with a 1000 cGy split dose from a gamma source, administered as two 500 cGy doses four hours apart. Post-irradiation, each mouse received a tail vein injection of 2×10⁶ freshly harvested donor bone marrow cells, with WT mice receiving LYST mutant bone marrow and LYST mutant mice receiving WT bone marrow. Four to five weeks post-transplantation, successful immunological reconstitution was confirmed by fluorescence-activated cell sorting (FACS) for CD45+ cells (AB_2734134, BD Biosciences) and PCR to determine the genotype of the sorted cells. After confirming successful reconstitution, mice were considered ready for TEVG implantation.

### Graft Implantation

TEVGs were implanted using microsurgical techniques. Briefly, mice were anesthetized with an intraperitoneal injection of ketamine (100 mg/kg) and xylazine (10 mg/kg) diluted in isotonic saline. A trained microsurgeon performed a midline laparotomy incision, the IVC and aorta were bluntly dissected, and the IVC was clamped on both proximal and distal sides with two microclamps. After obtaining vascular control, the IVC was transected, and the scaffold was implanted as an interposition graft. Throughout surgery, heparin (0.75 ml of 100 U/ml) was used to bathe the abdominal cavity and sites of anastomosis. Mice were administered post-operative analgesic for 48 hours (ibuprofen, 30 mg/kg, drinking water). Equal groups of male and female mice between 6-12 weeks of age were implanted (Table 3). A total of 255 animals were implanted with TEVGs across all study groups.

### Vascular Ultrasound

Ultrasonography was conducted to assess perioperative TEVG outcomes at day 3 post-implantation by examining graft diameter and venous flow. Mice were anesthetized with 1.5% inhaled isoflurane via a nose cone and placed on a heated platform. Using the Vevo3100 imaging system (FUJIFILM Sonosite, Inc., Tokyo, Japan), a trained investigator captured high-resolution B-mode, color doppler, and doppler images to assess graft morphology and blood flow dynamics. Data analysis was performed using VevoLAB software to ensure detailed assessment of graft structure and function, aiding in the evaluation of TEVG viability and performance.

### Micro-CT

Mice were positioned in restraining tubes to receive 100 μL injections of EXITRON® 12000 contrast agent through tail vein injection, and were subsequently sedated with 1.5% isoflurane/1 L/min Oxygen. The mice were placed on heated beds during the scan (40 μm resolution with a 360° rotation, 50 kVp voltage, 0.43 mA current, one projection per step with a 0.375° angle increment, 40 ms exposure time, and 1x1 binning, resulting in 960 projections). A fixed 100 μm aluminum filter and a 1.80 mm HE-UHR-RM collimator were used. Scans were performed using MILabs® 12.29 software with specific parameters: CT mode, Ultra-Focus magnification, single energy source, normal scan mode. Selected NII files were converted to DICOM using XMedcon® or Vivoquant® and uploaded for analysis. A trained investigator segmented the TEVG from the inflow to the outflow anastomosis. PMOD Technologies LLC (Zurich, Switzerland) software was used to calculate luminal diameter and volume.

To exclude artifactual narrowing at the anastomoses, measurements were standardized by focusing on the midgraft region, dividing the TEVG into 40 segments (40 μm thickness/each segment). This approach enabled comparisons of luminal diameter and volume within the central segment of the graft.

### Histology and Immunohistochemistry

Mice were euthanized at 3-days (n=3/WT, Global LYST mutant, and P2Y12 KO) and 14-days (n=224 including all study mice). An overdose of the anesthesia cocktail was administered into the right ventricle. After isolating the graft via midline laparotomy, the right atrium was transected, and the left ventricle was perfused with heparinized saline and 10% neutral buffered formalin (NBF). The graft was then removed and fixed in 10% NBF. Explanted grafts were stored in 10% NBF for 24 hours at 4° C., processed through graded alcohol, paraffin-embedded, and sectioned at 4 μm. Hematoxylin and eosin (H&E) and Carstair's stains were performed using standard procedures. For immunohistochemistry for CD31 and myosin heavy chain 11 (MYH11), slides were deparaffinized, rehydrated, and antigen retrieval was performed in citrate (pH 6.0) or tris-EDTA (pH 9.0), respectively, using a pressure cooker for 10 minutes. Endogenous peroxidases were blocked with 3% hydrogen peroxide, and nonspecific background with Background Sniper (Biocare Medical, Pacheco, CA) containing 3% goat serum. Sections were stained using primary antibodies (anti-CD31 for endothelial cells and anti-myosin heavy chain 11 for smooth muscle cells), followed by biotinylated goat anti-rabbit IgG, avidin-HRP, and chromogenic development with 3, 3′-diaminobenzidine (DAB). Slides were counterstained with Gill's hematoxylin followed by treatment with Scott's bluing solution and finally dehydrated before cover-slipping. Images were obtained using a ZEISS AXIO OBSERVER® Z1 inverted microscope, 89-North PhotoFluor LM-75 light source with appropriate filters and a ZEISS AXIOCAM® 105 (color) digital camera with 20× objectives.

### Scanning Electron Microscopy

Three scaffolds were implanted for 3 days, then explanted, rinsed with phosphate buffered saline (“PBS”), and fixed in 10% non-buffered formalin (“NBF”) overnight. After another PBS rinse, they were dehydrated in ethanol (50%, 70%, 80% for 5 minutes each; 95%, 100% for 10 minutes twice) and graded hexamethyldisilazane (“HMDS”) mixtures in ethanol (25%, 50%, 75%, 100% for 15 minutes each). The grafts were air-dried, bisected lengthwise, mounted with the luminal face exposed, and sputter-coated with 3.0 nm gold. Imaging was performed using a HITACHI S-4800® SEM at 5.0 kV and 10 μA.

### Statistical Analysis

Sample sizes (n) were selected using power analysis with an alpha of 0.05 and 80% power. All statistical analyses were performed using GraphPad Prism 9 or IBM SPSS Statistics. Critical stenosis was defined as a 75% decrease in luminal diameter, and occlusion was defined as complete obliteration of the lumen. Patency outcomes were measured as minimum luminal diameter and volume of the TEVG using Micro-CT. The flow cytometry and qPCR data were analyzed using FlowJo and StepOne software, respectively. All datasets were tested for normality using the Shapiro-Wilk test to determine appropriate comparison tests. Experiments with two groups were evaluated using Student's t-test, with Welch's correction applied where standard deviations differed between groups. Comparisons between more than two groups, such as for luminal diameter comparisons, were conducted using ANOVA followed by post-hoc tests and Dunn's multiple comparisons test. If any groups were non-normally distributed, significant differences between groups were assessed using the nonparametric Mann-Whitney U or Kruskal-Wallis test. A p-value<0.05 was considered statistically significant. The incidence of stenosis across groups was evaluated using Fisher's Exact Test, comparing numbers of patent and occluded TEVGs of experimental groups to WT.

### Results

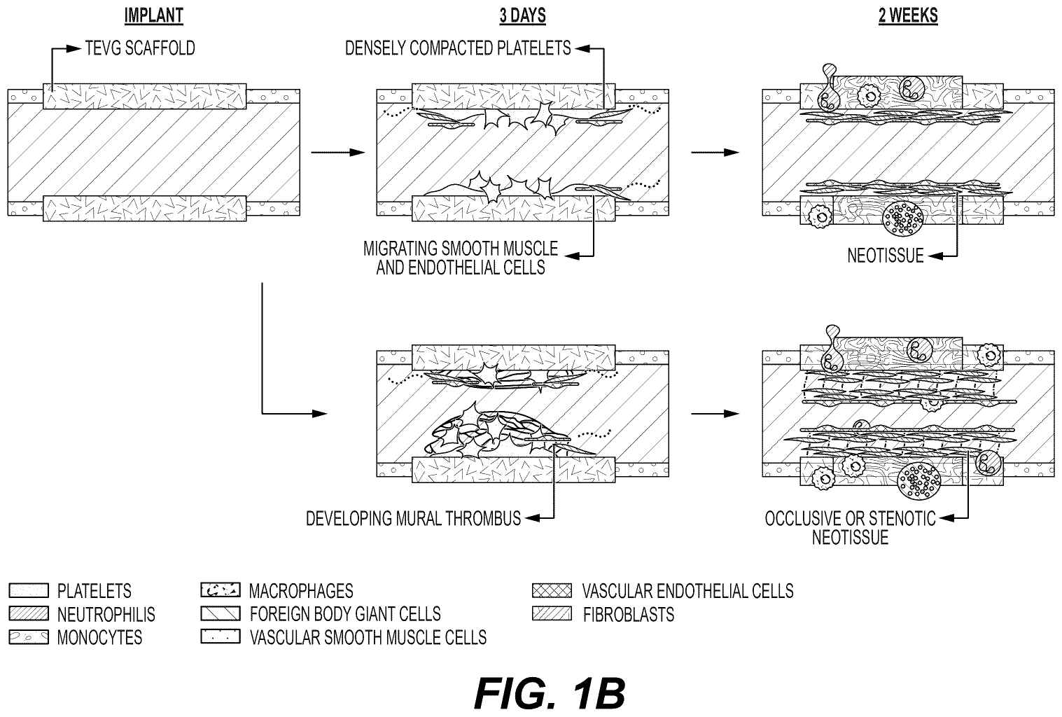

FIG. 2A is the time post TEVG transplant. FIG. 2B is a graph of the resulting minimum luminal diameter (mm) for a wild type versus LYST mutant (2B) and wild type versus LYST mutant receiving a bone marrow transplant (2C). 23/24 (96%) of TEVGs implanted into WT mice transplanted with LYST mutant bone marrow were widely patent (0.93±0.23 mm, n=24) compared to only 8/21 (38%) patent TEVGs in LYST mutant mice transplanted with WT bone marrow (0.34±0.44, n=22) (p<0.0001). (FIG. 2B) This observation confirmed the bone marrow-derived lineage of the cell type causing TEVG stenosis or occlusion.

LYST mutants are shown in FIG. 3A. FIG. 3B is a graph of the minimum luminal diameter (mm) for wild type (WT) and global LYST mutant following TEVG implantation. FIG. 3C is a graph of the minimum luminal diameter (mm) for wild type (WT), LYST Ex52 Floxed mutant, neutrophil LYST mutant and macrophage LYST mutant following TEVG implantation.

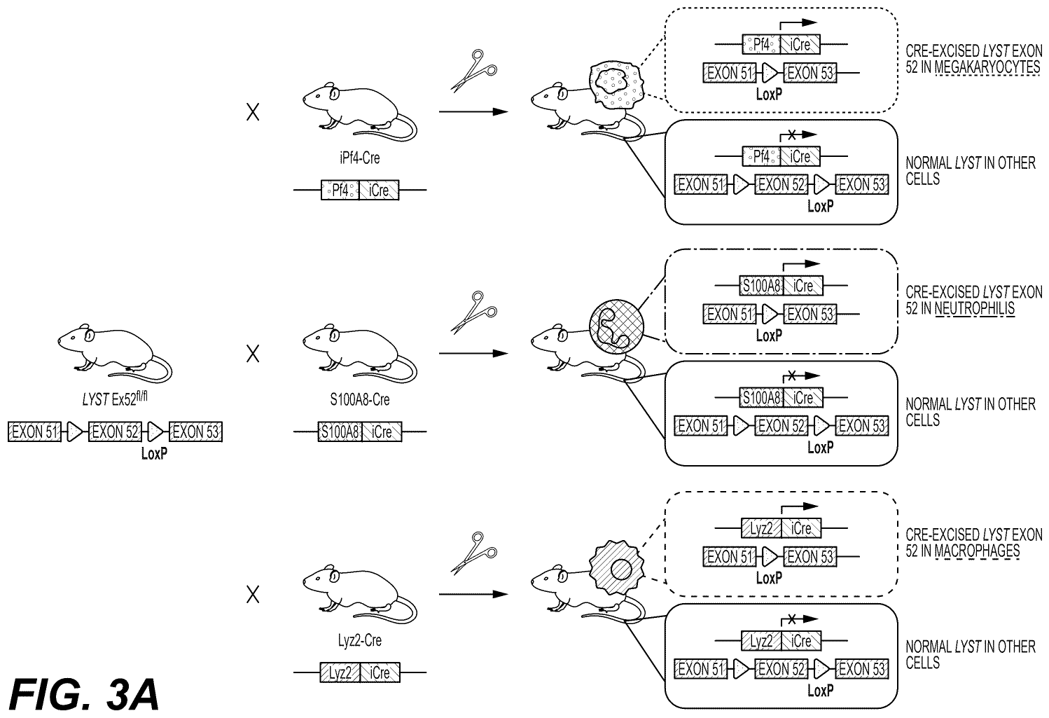

Comparing the minimum luminal diameter (mm) for wild type (WT), floxed, neutrophil LYST mutant, and macrophage LYST mutants (FIG. 3C), and global LYST mutant and platelet LYST mutant (FIG. 4A, FIG. 4B) showed that only the platelet LYST mutant significantly reduced the incidence of stenosis, with 97% remaining patent at the 2 week timepoint as opposed to only 25% patency in the WT group. The platelet count did not significantly different between WT, global LYST mutant and platelet LYST mutant. FIG. 4C.

Neutrophil and macrophage LYST mutants were not significantly different than negative controls. The platelet specific LYST mutant was sufficient to recapitulate the global phenotype, indicating that the LYST-mediated signaling leading to stenosis is initiated by platelets.

Only the platelet specific LYST mutation significantly reduced the incidence of stenosis, with 97% remaining patent at the 2 week timepoint as opposed to only 25% patency in the WT group. Neutrophil and macrophage LYST mutants were not significantly different than negative controls. The platelet specific LYST mutant was sufficient to recapitulate the global phenotype, indicating that the LYST-mediated signaling leading to stenosis is initiated by platelets.

LYST mutant platelets aggregate but do not release granules in response to thrombin. Platelets can also release their alpha granules, which contain protein molecules involved in coagulation and wound healing. In addition, they upregulate integrin receptors that facilitate adhesion and aggregation.LYST mutant platelets aggregate but do not release granules in response to thrombin or ADP. LYST is important for dense granule signaling but does not appreciably affect aggregation, alpha granule release or adherence. LYST mutations cause ineffective dense granule exocytosis yet preserve the ability of platelets to aggregate and adhere and release alpha granule contents. Platelets can also release their alpha granules, which contain protein molecules involved in coagulation and wound healing. In addition, they upregulate integrin receptors that facilitate adhesion and aggregation. Aberrant remodeling is initiated by platelet-mediated purinergic signaling.

LYST mutations do not affect alpha granule secretion (p-selectin) or integrin signaling (integrin αIIbβ3). Flow cytometry experiments demonstrated that in response to stimulation, LYST mutant platelets could still release alpha granules and upregulate integrin receptors, evidenced by the upregulation of p selectin and integrin alpha 2 beta 3 respectively. LYST mutant platelets still adhere to scaffolds. Supported by SEM and Carstairs staining demonstrating that by day three, LYST mutant platelets still adhered and could form a densely compacted provisional matrix that appeared to remodel into collagen rich neotissue by day 14.

This study determined which cell types and receptors are involved in initiating stenosis. To study the role of LYST mutations in aberrant TEVG remodeling, seeded and unseeded TEVGs were implanted into LYST Exon52 deletion mice (global LYST mutant) and C57BL/6J (WT) mice. The TEVGs were monitored using ultrasound at day 3 and Micro-CT at day 14. Ultrasound interrogation revealed that acute luminal narrowing occurred by day 3 through complete occlusion or severe stenosis. Ultrasound results were in general agreement with the 14-day Micro-CT scans. Micro-CT measurements demonstrated a bimodal distribution of luminal diameter, with most grafts either completely occluded or widely patent. Grafts noted as occluded on day 3 did not resolve by day 14 post-implantation, and patent grafts did not narrow beyond day 3. Accordingly, at the 14-day timepoint, 6/24 (25%) of WT TEVGs were patent, with 75% becoming occluded as evidenced by no identifiable graft by CT as well as significant collateral vessel development. Thus, the occluded grafts were designated luminal diameters of 0 mm. In contrast, 22/24 (92%) of global LYST mutants remained patent at the 14-day timepoint (average diameter 0.90±0.34 mm). The global LYST mutants had a statistically significant lower incidence of graft narrowing compared to the WT group (p<0.0001|Fisher's Exact Test).

Histological findings complemented Micro-CT data, showing that TEVGs in global LYST mutant mice exhibited broad patency and a neotissue-lined lumen, whereas most WT TEVGs displayed excessive luminal neotissue, leading to vessel occlusion. Patent WT grafts demonstrated similar findings as the global LYST mutants, yet most WT TEVGs showed excessive luminal neotissue, occluding the vessel. Separate groups of both WT and global LYST mutant mice were implanted to follow long-term patency and effects of the LYST mutation on neovessel formation. The TEVGs were explanted at 6 months (n=10/group) and at 2 years (n=1/group). By 6 months, within both WT and global LYST mutant mice, the scaffold had partially degraded and a neovessel formed with a neointima and neomedia. By 2 years, the scaffold had completely degraded, leaving behind a neovessel whose cellular makeup resembled the native vein.

The results demonstrate that TEVG stenosis/occlusion is a complex multistage process that is initiated by LYST-mediated platelet function.

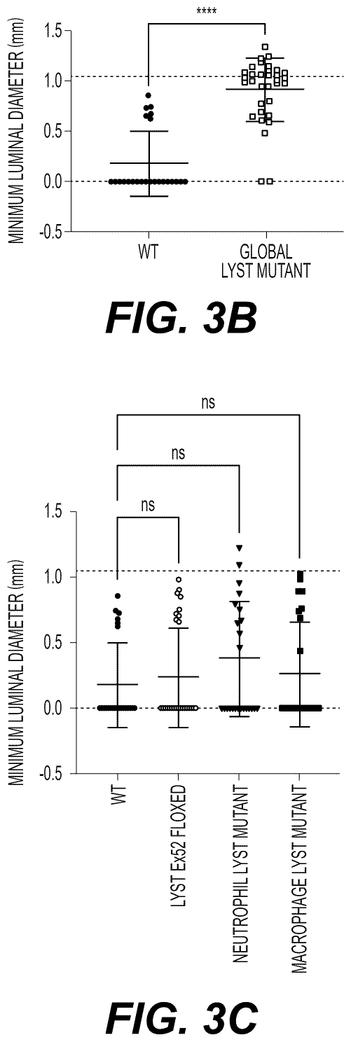

Focusing on early infiltrators in the foreign body response to the TEVG, various cell-specific LYST mutants were generated to ascertain the cell-specific contributions of LYST to TEVG stenosis or occlusion (Table 1). It was found that neither the neutrophil (10/23 (43%) patent) nor the macrophage specific LYST mutants (10/29 (34%) patent) affected 3- or 14-day TEVG outcomes compared to WT controls (p=0.227 and p=0.554, respectively). In the Lyst Ex52^{fl/fl} control group, which harbor the LoxP sites with no Cre present, 9/28 (32%) of TEVGs were patent, with an incidence of narrowing that was not significantly different than the WT incidence (p=0.760|Fisher's Exact Test). This indicates that the presence of LoxP sites alone does not influence the narrowing outcomes in TEVGs. The platelet specific LYST mutant showed significantly improved TEVG patency (29/30 (96.6%) patent) compared to WT controls (p<0.0001 (Fisher's Exact Test) and did not vary significantly from the global LYST mutant (p=0.592). Platelet counts were not significantly different between strains. Additionally, Pf4-Cre controls without the LoxP sites did not experience improved patency rates compared to WT (6/16 (37%) patent) (p=1.0|Fisher's Exact Test), demonstrating that disrupting platelet specific LYST function was responsible for preventing stenosis. FIG. 5.

LYST mutant platelets can still adhere to biomaterial and aggregate but cannot effectively release dense granule contents, including ADP and ATP. In collagen stimulated platelets, α-granule mobilization (P-selectin expression) and integrin signaling (Integrin αIIbβ3 expression) was unaffected by the LYST mutation. Neither the global LYST mutant nor the platelet specific LYST mutant showed significantly different platelet aggregation compared to WT platelets after stimulation with thrombin (1 U/mL) or ADP (5 mg/mL) (p=0.8829 and p=0.2746, respectively, one-way ANOVA with Dunn's multiple comparisons). However, the LYST mutant platelets displayed impaired aggregation in response to collagen stimulation (5 mg/mL) (p<0.0001). The impaired aggregation was overcome by increasing the collagen concentration (20 mg/mL). Consistently, the global and platelet specific LYST mutants had impaired dense granule exocytosis (ATP release) in response to thrombin and collagen (p<0.0001). Platelets did not release dense granules in response to ADP stimulus alone regardless of genotype.

Gross visualization of the grafts explanted at day 3 revealed that all TEVGs explanted from LYST mutants had a densely compacted layer of platelets on the luminal surface. Some areas displayed networks of fibrin that trapped circulating erythrocytes. Histological sections stained using Carstair's method to distinguish fibrin and platelets identified platelets as the highly compacted layer on the surface of the scaffold. Each TEVG explanted from WT mice contained a semi- or completely occlusive thrombus, primarily composed of platelets and fibrin.

It was found that only the platelet specific LYST mutation significantly reduced the incidence of stenosis, with 97% remaining patent at the 2 week timepoint as opposed to only 25% patency in the WT group.

FIGS. 3B and 4A-4C are graphs of the minimum luminal diameter (mm) for wild type (WT), floxed, global LYST mutant, platelet LYST mutant, and macrophage LYST mutants, showing that only the platelet LYST mutant significantly reduced the incidence of stenosis.

Neutrophil and macrophage LYST mutants were not significantly different than negative controls. The platelet specific LYST mutant was sufficient to recapitulate the global phenotype, indicating that the LYST-mediated signaling leading to stenosis is initiated by platelets.

Only the platelet specific LYST mutation significantly reduced the incidence of stenosis, with 97% remaining patent at the 2 week timepoint as opposed to only 25% patency in the WT group. Neutrophil and macrophage LYST mutants were not significantly different than negative controls. The platelet specific LYST mutant was sufficient to recapitulate the global phenotype, indicating that the LYST-mediated signaling leading to stenosis is initiated by platelets.

The roles of the various factors in aggregation, granule secretion and adhesion are shown in FIGS. 1A and 1B. LYST mutant platelets aggregate but do not release granules in response to thrombin or ADP. Platelets can also release their alpha granules, which contain protein molecules involved in coagulation and wound healing. In addition, they upregulate integrin receptors that facilitate adhesion and aggregation. LYST mutant platelets aggregate but do not release granules in response to thrombin or ADP.

LYST is important for dense granule signaling but does not appreciably affect aggregation, alpha granule release or adherence. LYST mutations cause ineffective dense granule exocytosis yet preserve the ability of platelets to aggregate and adhere and release alpha granule contents. Platelets can also release their alpha granules, which contain protein molecules involved in coagulation and wound healing. In addition, they upregulate integrin receptors that facilitate adhesion and aggregation. Aberrant remodeling is initiated by platelet-mediated purinergic signaling.

LYST mutations do not affect alpha granule secretion (p-selectin) or integrin signaling (integrin αIIbβ3). Flow cytometry experiments demonstrated that in response to stimulation, LYST mutant platelets could still release alpha granules and upregulate integrin receptors, evidenced by the upregulation of p selectin and integrin alpha 2 beta 3 respectively. LYST mutant platelets still adhere to scaffolds. Supported by SEM and Carstairs staining demonstrating that by day three, LYST mutant platelets still adhered and could form a densely compacted provisional matrix that appeared to remodel into collagen rich neotissue by day 14.

LYST is important for effective dense granule signaling but does not appreciably affect aggregation, alpha granule release or adherence. LYST mutations cause ineffective dense granule exocytosis yet preserve the ability of platelets to aggregate and adhere and release alpha granule contents.

Aberrant remodeling is initiated by platelet-mediated purinergic signaling. It is therefore a balance to prevent inadequate or delayed neotissue formation as well as aberrant remodeling. Platelets appear to exert control over tissue regeneration, acting not as just responders but regulators of remodeling. Platelets promote regeneration. In the vascular conduit, platelets appear to exert control over tissue regeneration, acting not as just responders but regulators of remodeling. Too little platelet activity results in inadequate neotissue formation while excessive platelet aggregation facilitates aberrant remodeling. Modulating aggregation appears to influence long-term outcomes. Overall, strategically controlling the immune microenvironment at the site of implantation can lead to favorable tissue regeneration across a range of clinical contexts.

Comparing the minimum luminal diameter (mm) for wild type (WT), floxed, global LYST mutant, platelet LYST mutant, and macrophage LYST mutants, showed that only the platelet LYST mutant significantly reduced the incidence of stenosis, with 97% remaining patent at the 2-week timepoint as opposed to only 25% patency in the WT group. Neutrophil and macrophage LYST mutants were not significantly different than negative controls. The platelet specific LYST mutant was sufficient to recapitulate the global phenotype, indicating that the LYST-mediated signaling leading to stenosis is initiated by platelets.

Only the platelet specific LYST mutation significantly reduced the incidence of stenosis, with 97% remaining patent at the 2 week timepoint as opposed to only 25% patency in the WT group. Neutrophil and macrophage LYST mutants were not significantly different than negative controls. The platelet specific LYST mutant was sufficient to recapitulate the global phenotype, indicating that the LYST-mediated signaling leading to stenosis is initiated by platelets.

LYST mutant platelets aggregate but do not release granules in response to thrombin. Platelets can also release their alpha granules, which contain protein molecules involved in coagulation and wound healing. In addition, they upregulate integrin receptors that facilitate adhesion and aggregation. LYST mutant platelets aggregate but do not release granules in response to thrombin or ADP. LYST is important for dense granule signaling but does not appreciably affect aggregation, alpha granule release or adherence. LYST mutations cause ineffective dense granule exocytosis yet preserve the ability of platelets to aggregate and adhere and release alpha granule contents. Platelets can also release their alpha granules, which contain protein molecules involved in coagulation and wound healing. In addition, they upregulate integrin receptors that facilitate adhesion and aggregation. Aberrant remodeling is initiated by platelet-mediated purinergic signaling.

LYST mutations do not affect alpha granule secretion (p-selectin) or integrin signaling (integrin αIIbβ3). Flow cytometry experiments demonstrated that in response to stimulation, LYST mutant platelets could still release alpha granules and upregulate integrin receptors, evidenced by the upregulation of p selectin and integrin alpha 2 beta 3 respectively. LYST mutant platelets still adhere to scaffolds. By day three, LYST mutant platelets still adhered and could form a densely compacted provisional matrix that appeared to remodel into collagen rich neotissue by day 14.

### Summary and Significance

This study determined which cell types and receptors are involved in initiating stenosis. To study the role of LYST mutations in aberrant TEVG remodeling, seeded and unseeded TEVGs were implanted into LYST Exon52 deletion mice (global LYST mutant) and C57BL/6J (WT) mice. The TEVGs were monitored using ultrasound at day 3 and Micro-CT at day 14.

In untreated animals, ultrasound interrogation revealed that acute luminal narrowing occurred by day 3 through complete occlusion or severe stenosis. Ultrasound results were in general agreement with the 14-day Micro-CT scans. Micro-CT measurements demonstrated a bimodal distribution of luminal diameter, with most grafts either completely occluded or widely patent. Grafts noted as occluded on day 3 did not resolve by day 14 post-implantation, and patent grafts did not narrow beyond day 3. At the 14-day timepoint, 6/24 (25%) of WT TEVGs were patent, with 75% becoming occluded as evidenced by no identifiable graft by CT as well as significant collateral vessel development. The occluded grafts were designated luminal diameters of 0 mm. In contrast, 22/24 (92%) of global LYST mutants remained patent at the 14-day timepoint (average diameter 0.90±0.34 mm). The global LYST mutants had a statistically significant lower incidence of graft narrowing compared to the WT group (p<0.0001|Fisher's Exact Test).

Histological findings complemented Micro-CT data, showing that TEVGs in global LYST mutant mice exhibited broad patency and a neotissue-lined lumen, whereas most WT TEVGs displayed excessive luminal neotissue, leading to vessel occlusion. Patent WT grafts demonstrated similar findings as the global LYST mutants, yet most WT TEVGs showed excessive luminal neotissue, occluding the vessel. Separate groups of both WT and global LYST mutant mice were implanted to follow long-term patency and effects of the LYST mutation on neovessel formation. The TEVGs were explanted at 6 months (n=10/group) and at 2 years (n=1/group). By 6 months, within both WT and global LYST mutant mice, the scaffold had partially degraded and a neovessel formed with a neointima and neomedia. By 2 years, the scaffold had completely degraded, leaving behind a neovessel whose cellular makeup resembled the native vein.

TEVG stenosis/occlusion is a complex multistage process that is initiated by LYST-mediated platelet function. A reciprocal bone marrow transplant (BMT) model was used to evaluate whether the cell type responsible for initiating LYST-mediated TEVG stenosis originates in the bone marrow. Recipient mice (WT or global LYST mutant; n=24/each) were irradiated prior to injection of donor bone marrow cells from either LYST mutant or WT mice. The high dose of radiation allowed for nearly complete reconstitution of the recipient immune system with donor cells. Five weeks after BMT, TEVGs were implanted into the transplanted mice and were explanted 14 days later. Diameters were obtained using in vivo Micro-CT scans. 23/24 (96%) of TEVGs implanted into WT mice transplanted with LYST mutant bone marrow were widely patent (0.93±0.23 mm, n=24) compared to only 8/21 (38%) patent TEVGs in LYST mutant mice transplanted with WT bone marrow (0.34±0.44, n=22) (p<0.0001). This observation confirmed the bone marrow-derived lineage of the cell type causing TEVG stenosis or occlusion.

Focusing on early infiltrators in the foreign body response to the TEVG, various cell-specific LYST mutants were generated to ascertain the cell-specific contributions of LYST to TEVG stenosis or occlusion (Table 1). It was found that neither the neutrophil (10/23 (43%) patent) nor the macrophage specific LYST mutants (10/29 (34%) patent) affected 3- or 14-day TEVG outcomes compared to WT controls (p=0.227 and p=0.554, respectively). In the Lyst Ex52^{fl/fl} control group, which harbor the LoxP sites with no Cre present, 9/28 (32%) of TEVGs were patent, with an incidence of narrowing that was not significantly different than the WT incidence (p=0.760|Fisher's Exact Test). This indicates that the presence of LoxP sites alone does not influence the narrowing outcomes in TEVGs. The platelet specific LYST mutant showed significantly improved TEVG patency (29/30 (96.6%) patent) compared to WT controls (p<0.0001 (Fisher's Exact Test) and did not vary significantly from the global LYST mutant (p=0.592). Platelet counts were not significantly different between strains. Additionally, Pf4-Cre controls without the LoxP sites did not experience improved patency rates compared to WT (6/16 (37%) patent) (p=1.0 Fisher's Exact Test), demonstrating that disrupting platelet specific LYST function was responsible for preventing stenosis.

LYST mutant platelets can still adhere to biomaterial and aggregate but cannot effectively release dense granule contents, including ADP and ATP

In collagen stimulated platelets, α-granule mobilization (P-selectin expression) and integrin signaling (Integrin αIIbβ3 expression) were unaffected by the LYST mutation. Neither the global LYST mutant nor the platelet specific LYST mutant showed significantly different platelet aggregation compared to WT platelets after stimulation with thrombin (1 U/mL) or ADP (5 mg/mL) (p=0.8829 and p=0.2746, respectively, one-way ANOVA with Dunn's multiple comparisons). However, the LYST mutant platelets displayed impaired aggregation in response to collagen stimulation (5 mg/mL) (p<0.0001). The impaired aggregation was overcome by increasing the collagen concentration (20 mg/mL). Consistently, the global and platelet specific LYST mutants had impaired dense granule exocytosis (ATP release) in response to thrombin and collagen (p<0.0001). Platelets did not release dense granules in response to ADP stimulus alone regardless of genotype.

Gross visualization of the grafts explanted at day 3 revealed that all TEVGs explanted from LYST mutants had a densely compacted layer of platelets on the luminal surface. Some areas displayed networks of fibrin that trapped circulating erythrocytes. Histological sections stained using Carstair's method to distinguish fibrin and platelets identified platelets as the highly compacted layer on the surface of the scaffold. Each TEVG explanted from WT mice contained a semi- or completely occlusive thrombus, primarily composed of platelets and fibrin.

Ultrasound and Micro-CT analysis revealed that TEVGs were widely patent at day 3 and 100% of TEVGs remained widely patent by day 14 (1.0±0.12 mm) (p<0.0001|Fisher's Exact Test). Similar to patent WT TEVGs, PRASUGREL-treated mice exhibited neotissue formation characterized by an endothelial cell-lined lumen (CD31) surrounded by subendothelial layers of smooth muscle cells (MyH11), resembling native veins. This finding demonstrates that PRASUGREL treatment did not affect luminal endothelialization at 14 days.

Cells that contribute to the early foreign body response to TEVGs are shown in FIG. 1A, B. Unseeded TEVGs were implanted into Lyst Exon 52 deletion mice (global Lyst mutant) and C57BL/6J (WT) mice. The TEVGs were monitored using ultrasound at day 3 and Micro-CT at day 14. Ultrasound interrogation revealed that acute luminal narrowing occurred by day 3 through complete occlusion or severe stenosis. Ultrasound results were in general agreement with the 14-day Micro-CT scans. Micro-CT measurements demonstrated a bimodal distribution of luminal diameter, with most grafts either completely occluded or widely patent. Within patent global Lyst mutant grafts, the Gaussian Mixture Model identified two components with means for minimum luminal diameter values at 1.07 mm (76.65%) and 0.68 mm (23.35%). Hartigan's Dip Test analysis indicated no significant deviation from unimodality (p=0.419). The bimodality coefficient (BC) did not exceed the threshold of 0.555 that would support a bimodal distribution (BC=0.507). Grafts noted as occluded on day 3 did not resolve by day 14 post-implantation, and patent grafts did not severely stenose beyond day 3. Accordingly, at the 14-day timepoint, 6/24 (25%) WT TEVGs were patent, with 75% becoming occluded as evidenced by no identifiable graft by Micro-CT as well as significant collateral vessel development. All occluded grafts were designated luminal diameters of 0 mm.

In contrast, 22/24 (92%) global Lyst mutants remained patent at the 14-day timepoint (average diameter 0.90±0.34 mm). The global Lyst mutants had significantly lower incidence of graft narrowing compared to the WT group (p<0.0001|Fisher's Exact Test) (FIGS. 4A, 4B). Histological findings complemented Micro-CT data, showing that TEVGs in global Lyst mutant mice exhibited broad patency and a neotissuelined lumen, whereas most WT TEVGs displayed excessive luminal neotissue that ultimately occluded the vessel. Separate groups of both WT and global Lyst mutant mice were implanted to follow long-term patency and effects of the Lyst mutation on neovessel formation. The TEVGs were explanted at 6 months (n=10/group) and at 2 years (n=1/group). By 6 months, within both WT and global Lyst mutant mice, the scaffold had partially degraded, and a neovessel formed with a neointima and neomedia. By 2 years, the scaffold had completely degraded, leaving behind a neovessel with structural architecture resembling the native vein.

### Summary

A mutation in the lysosomal trafficking regulator gene (LYST) alters the acute foreign body response, reducing TEVG stenosis without compromising neovessel formation. LYST is a widely expressed and evolutionarily conserved protein that influences a spectrum of biological processes involved in innate immunity such as degranulation. LYST mutations cause impaired immune function, bleeding tendency, and abnormal intracellular transport to and from lysosomes. In the present study, macrophage and neutrophil specific LYST mutations failed to prevent stenosis, suggesting that macrophages and neutrophils do not elicit the initial signal causing stenosis. Conversely, disrupting LYST-mediated platelet function significantly decreased stenosis to rates observed in the global LYST mutants. This indicated platelets as early orchestrators of the acute foreign body response to the scaffold that, if uncontrolled, could lead to vessel narrowing.

LYST mutant platelets exhibit normal aggregation in response to ADP and thrombin, yet demonstrate impaired dense granule secretion, preventing the release of molecules such as ADP, ATP, and calcium. This dysfunction prevents proper amplication of the activation signal, which could explain why the LYST mutant platelets displayed impaired aggregation in response to collagen. The inability of LYST mutant platelets to effectively release dense granules likely modulates platelet aggregation in vivo. While dense granule exocytosis was diminished, stimulated LYST mutant platelets still exposed p-selectin, a marker of alpha granule release, and expressed high affinity integrin αIIβIII, which binds fibrinogen and supports platelet aggregation. Despite the impaired dense granule release, LYST mutant platelets still adhered to the TEVG scaffold, forming a compacted platelet-rich layer by day 3 that remodeled into neotissue by day 14. The dense granule defect with other platelet functions intact appeared to moderate the acute foreign body response, modulating aggregation without hindering the necessary processes that prevent hemorrhage, establish a provisional matrix, and facilitate ingrowth of endothelial and smooth muscle cells.

LYST is important for dense granule signaling but does not appreciably affect aggregation, alpha granule release or adherence. LYST mutations cause ineffective dense granule exocytosis yet preserve the ability of platelets to aggregate and adhere and release alpha granule contents. Platelets can also release their alpha granules, which contain protein molecules involved in coagulation and wound healing. In addition, they upregulate integrin receptors that facilitate adhesion and aggregation. Aberrant remodeling is initiated by platelet-mediated purinergic signaling.

Platelets appear to exert control over tissue regeneration in vascular conduits, acting not as just responders but regulators of remodeling. Too little platelet activity results in inadequate neotissue formation while excessive platelet aggregation facilitates aberrant remodeling. Modulating aggregation and thereby strategically controlling the immune microenvironment at the site of implantation can lead to favorable tissue regeneration across a range of clinical contexts.

ADP released from dense granules supports proper thrombus formation by reinforcing platelet activation and aggregation through the P2Y12 receptor. However, ADP interactions with P2Y12 are not necessarily required for platelet adherence to blood proteins adsorbed to the luminal TEVG surface. Given that ADP potentiates sustained platelet aggregation via the P2Y12 receptor, inhibiting this pathway could reduce stenosis by interfering with thrombus stabilization. Experiments in P2Y12 knockout and PRASUGREL-treated mice revealed that neither group developed stenosis post-implantation. Importantly, PRASUGREL effectively inhibited stenosis while preserving vascular neotissue formation at 14-days. This finding shows that inhibiting ADP-mediated platelet signaling could prevent aberrant remodeling without hindering vascular neotissue formation.

To disrupt the acute platelet signaling that initiates stenosis, the therapeutic must be administered prior to surgery to ensure that all platelets were exposed to the agent. These findings highlight the importance of timing and targeted modulation of platelet function to balance the need for platelet-mediated healing while preventing excessive neotissue formation.

Currently, in the clinical setting, TEVGs are implanted as scaffolds seeded with autologous bone marrow cells. Initially, bone marrow cell seeding was implemented as a strategy to accelerate endothelization promoting early healing. However, it was found that cell seeding was not required for neovessel formation but rather reduced stenosis in a dose-dependent manner and reduced macrophage infiltration into the scaffold. In clinical trials, a number of patients implanted with seeded scaffolds still developed stenosis. Further investigations revealed that seeded bone marrow cells had antiplatelet effects, suggesting that the seeded cells may reduce stenosis by disrupting the hemostatic response, potentially preventing the accumulation of ADP in the TEVG microenvironment and altering the subsequent immune response. As demonstrated in this study, antiplatelet agents like PRASUGREL help balance the acute foreign body response, which facilitates the development of an off-the-shelf TEVG and abrogate the need for bone marrow cell seeding. This approach simplifies production and broaden access to TEVGs for cardiovascular treatments, especially for facilities without specialized cell-seeding capabilities.

ADP-mediated platelet signaling appears to be the initial signal or a necessary cofactor that leads to stenosis. However, wound healing and remodeling of the provisional matrix into neotissue still requires the function of other cells, indicating that immune cells other than platelets can also influence TEVG outcomes. Since the neutrophil and macrophage LYST mutants did not affect the incidence of stenosis, their roles in stenosis and resolution of stenosis appear to either be independent of LYST function or act downstream of the platelet signaling. Macrophage depletion prohibits neotissue formation, yet in macrophage depleted models, platelet adherence and aggregation can still occur. Considering that LYST mutant macrophage models do not affect stenosis, it may be that while provisional matrix formation is necessary to initiate neotissue formation, macrophage function is essential for remodeling the initial thrombus into functional neotissue. Upon activation, platelets release many proteins and small molecules that act as inflammatory mediators and chemoattractants. ADP itself not only amplifies platelet aggregation but has also been shown to accelerate wound healing and fibroblast proliferation. While there are many molecules involved in wound healing, transforming growth factor-beta (TGF-β) and platelet derived growth factor (PDGF) have well documented roles in vascular repair. TGF-β, released by both platelets and other immune cells, could enhance platelet ADP reactivity and influence immune responses during and downstream of provisional matrix formation. TGF-β could also be involved in the crosstalk of platelets with subsequent infiltrating cells, modulating processes such as the transition of monocytes to macrophages, smooth muscle and endothelial cell proliferation and migration, or extracellular matrix remodeling. Similarly, PDGF was found to contribute to vascular neotissue formation by regulating smooth muscle cell proliferation, and extracellular matrix deposition. It is believed that the transient development of a provisional matrix on the TEVG is the first step in neotissue development. Then, the provisional matrix acts as significant source of cytokines and growth factors, such as TGF-β and PDGF, that facilitate further immune interactions and neovessel formation.

TEVGs serve as a model for understanding autologous tissue regeneration and the interplay between the immune and biomechanical factors that drive remodeling. In situ tissue engineering capitalizes on the body's own healing mechanisms to drive regeneration and remodeling at the site of implantation while allowing for external influence through various medical and/or procedural interventions. Furthermore, while TEVG is used for cardiovascular applications, this in situ approach is broadly applicable to other tissue engineering strategies including bone, soft tissue, and valvular structures where scaffolds leverage immune and biomechanical stimuli for effective regeneration. These models demonstrate that immune cells are not just responders but active regulators of tissue remodeling. Strategically controlling the immune microenvironment at the site of implantation can enhance the quality of tissue regeneration across a range of clinical contexts.

Following TEVG implantation in mice, platelets adhere to the scaffold, forming a layer of densely packed platelets, or develop into an occlusive, platelet-rich thrombus. Over two weeks, the resultant provisional matrix remodels into collagen-rich vascular neotissue including a neointima and neomedia through the recruitment and activity of other cells.

The findings in Example 1 demonstrate that a mutation in the lysosomal trafficking regulator gene (LYST) alters the acute foreign body response, reducing TEVG stenosis without compromising neovessel formation. LYST is a widely expressed and evolutionarily conserved protein that influences a spectrum of biological processes involved in innate immunity such as degranulation. LYST mutations cause impaired immune function, bleeding tendency, and abnormal intracellular transport to and from lysosomes.

In the present study, macrophage and neutrophil specific LYST mutations failed to prevent stenosis, suggesting that macrophages and neutrophils do not elicit the initial signal causing stenosis. Conversely, disrupting LYST-mediated platelet function significantly decreased stenosis to rates observed in the global LYST mutants. This indicated platelets as early orchestrators of the acute foreign body response to the scaffold that, if uncontrolled, could lead to vessel narrowing.

LYST mutant platelets exhibit normal aggregation in response to ADP and thrombin, yet demonstrate impaired dense granule secretion, preventing the release of molecules such as ADP, ATP, and calcium. This dysfunction prevents proper amplication of the activation signal, which could explain why the LYST mutant platelets displayed impaired aggregation in response to collagen. The inability of LYST mutant platelets to effectively release dense granules likely modulates platelet aggregation in vivo. While dense granule exocytosis was diminished, stimulated LYST mutant platelets still exposed p-selectin, a marker of alpha granule release, and expressed high affinity integrin αIIβIII, which binds fibrinogen and supports platelet aggregation.

Despite the impaired dense granule release, LYST mutant platelets still adhered to the TEVG scaffold, forming a compacted platelet-rich layer by day 3 that remodeled into neotissue by day 14. The dense granule defect with other platelet functions intact appeared to moderate the acute foreign body response, modulating aggregation without hindering the necessary processes that prevent hemorrhage, establish a provisional matrix, and facilitate ingrowth of endothelial and smooth muscle cells.

This study demonstrates that platelets are the initiators of LYST-mediated TEVG stenosis. It was determined that stenosis results from uncontrolled platelet aggregation ensuing from the acute foreign body response to the scaffold. The connection between P2Y12 and Lyst appears to be significant, albeit complex. Both P2Y12 and Lyst play critical roles in platelet function and thrombosis, though they act through distinct mechanisms. P2Y12 is involved in ADP-mediated signaling pathways, promoting platelet activation and aggregation, while Lyst regulates lysosomal trafficking and dense granule secretion, which affects the release of ADP and other bioactive molecules essential for hemostasis and thrombosis. Despite differences in mechanism, both pathways converge in modulating ADP-mediated platelet function. This overlap indicate that Lyst mutations impair platelet aggregation by indirectly limiting ADP availability, similar to how P2Y12 inhibition prevents ADP-mediated platelet function by limiting receptor activation.

### Example 2: Effect of Prasugrel on TEVG Stenosis/Occlusion in P2Y12 Knock Out Mice and Wild Type Mice

Based on the results obtained in Example 1 using LYST mutants, Prasugrel was tested on P2Y12 knock out (“KO”) mice and wild type (“WT”) mice to further investigate the role of ADP-mediated platelet signaling and the role of the P2Y12 receptor.

The pharmacological approach uses Prasugrel to inhibit purinergic signaling through the P2Y12 receptor. The genetic approach uses P2Y12 KO to remove purinergic signaling involved in ADP-mediated platelet aggregation.

When platelets become activated at the TEVG surface, they secrete ADP through degranulation, which forms a positive feedback loop that promotes further aggregation and thrombus growth. Prasugrel inhibits stenosis by interfering with this sustained platelet aggregation, which reduces the size of the developing thrombus. Due to decreased aggregation, there are fewer platelets degranulating and therefore decreased local concentrations of platelet-derived inflammatory mediators, chemokines, and growth factors. Therefore, P2Y12 inhibition may limit further cell infiltration as well as reduce the immunomodulatory effects platelets have on other cells.

P2Y12 has been detected in monocytes, macrophages, neutrophils, and endothelial cells, suggesting other immune cells may respond to ADP. It is unclear which monocyte or macrophage subtypes express P2Y12 and what affects their expression patterns. The general consensus is that P2Y12 signaling in monocytes and macrophages seems to be pro-healing or pro-fibrotic.

While other cells express P2Y12, the primary signaling occurs through platelets, and P2Y12 stimulation increases the platelet surface expression of other receptors involved in platelet function. P2Y12 inhibition may indirectly or directly impact the following processes: immune cell infiltration; skewing cells toward pro-inflammatory phenotypes or promoting inflammatory cytokine release; and fibrosis

### Materials and Methods

Unseeded TEVGs were implanted into P2Y12 KO mice which carry a global deletion of the P2Y12 receptor that binds to ADP and potentiates platelet activation. P2Y12 KO mice were received from Dr. Ania Majewska (University of Rochester, NY).

To determine whether pharmacological inhibition of P2Y12 would prevent stenosis, PRASUGREL was administered to WT mice two hours prior to TEVG implantation (n=14).

### Animal Care and Ethics Statement

All animals received humane care in compliance with the National Institutes of Health guide for the care and use of laboratory animals. The institutional animal care and use committee of the Abigail Wexner Research Institute at Nationwide Children's Hospital approved and monitored the care and use of all mice described in this report. Syngeneic 6-12-week-old female and male c57bl/6j (wt) mice (n=71) were purchased from Jackson Laboratories (strain #000664, Bar Harbor, ME) and were either TEVG recipients (n=66) or bone marrow donors (n=5). All mice were housed in a specific pathogen-free facility and kept in a temperature-controlled room set to a light and dark cycle of 12 h each. mice were provided with ad libitum access to standard mouse chow and water.

### TEVG Fabrication

Scaffolds were constructed from a biodegradable polymer felt with co-polymer sealant solution. Briefly, a 5 mm×7 mm rectangle of non-woven polyglycolic acid (PGA) felt (confluent medical, Warwick, RI) was wrapped around a 20 g stainless steel needle inserted within a polypropylene cylinder. using positive pressure, the PGA felt was then infused with a 5% (w/v) 50:50 copolymer of ε-caprolactone and l-lactide (PCLA) [Gunze, Ltd., Japan]. The tubularized scaffold, was snap-frozen at −80° C. for 30 min, then lyophilized for 24 h causing the PCLA to form a sponge-like network throughout the PGA felt. Afterward, scaffolds were removed from their molds and trimmed to 3 mm in length, the resulting scaffolds possessed an inner diameter of ˜0.91 mm and an average wall thickness of ˜300 μm. The scaffolds were stored in a desiccator for a minimum of 3 days. the day prior to implantation, scaffolds were sterilized by immersion in 70% ethanol followed by exposure to uv light for 15 min.

### BM-MNC Seeding of TEVGs

Wild-type mice were euthanized with an overdose of isoflurane followed by cervical dislocation as a secondary method of euthanasia. Bone marrow was harvested from the femurs, tibias, and pelvis by flushing marrow cavities with rpmi 1640. BM-MNCs were then isolated using density-gradient centrifugation by layering diluted bm over histopaque (1.08.3 g/cc). Cells obtained from the buffy coat were washed twice in 1×pbs then resuspended in RPMI 1640 with 1% penicillin/streptomycin (p/s) at a concentration of 750×10⁶ cells/ml. prior to seeding cells, scaffolds were prewet with 4 μl of PBS for 5 min. PBS was then aspirated and 4 μl of the BM-MNC solution containing 3.0×10⁶ cells was pipetted into the lumen of each scaffold. after static incubation at room temperature for 10 min, a 22 g needle was inserted into the lumen to provide negative buoyancy during overnight incubation. BM-MNC seeded and unseeded scaffolds were incubated overnight in 1 ml of RPMI 1640 with 1% P/S at 37° C. and 5% CO₂.

### Surgical Procedures

TEVG scaffolds were implanted into wt mice as infrarenal abdominal IVC interposition grafts. briefly, mice were anesthetized with 100 mg/kg ketamine, 10 mg/kg xylazine, and 5 mg/kg ketoprofen by intraperitoneal injection. a midline laparotomy incision was performed, the IVC and aorta were bluntly dissected, and the IVC was clamped on both proximal and distal sides with two micro-clamps. After obtaining vascular control, the IVC was transected and the TEVG scaffold was implanted using 10-0 nylon suture. throughout surgery, anticoagulation was provided by 0.2 ml of 100 μ/ml unfractionated heparin (Mylan Institutional, LLC) by bathing the abdominal cavity and anastomosis sites. after completion of the proximal and distal anastomoses, the vascular clamps were released allowing blood to flow through the scaffold. If blood hemorrhaged through the scaffold or at anastomotic sites, the IVC was reclamped and surgicel (Ethicon, Inc.) was applied to encourage hemostasis.

This process was repeated as necessary until no further bleeding was observed. Postoperatively, mice were given 30 mg/kg ibuprofen in drinking water for 48 hours for pain. Aside from the experimental design, no additional antiplatelet or anti-coagulation therapy was provided.

### Drug Treatment

PRASUGREL in pill form was crushed and suspended in 0.5% methyl cellulose (Sigma) at concentrations of 0.1, 0.3, or 1.0 mg/ml. mice were dosed twice with approximately 0.2 ml by oral gavage the day before and morning prior to scaffold implantation targeting doses of 1, 3, and 10 mg/kg, respectively. PRASUGREL is a third-generation thienopyridine that is more potent, faster acting, and lasts longer after discontinuation of treatment than clopidogrel. Mice were given 2 μg (0.1 ml, male-80 mu/g, female-100 mμ/g) intravenously at the start of surgery, prior to transection of the abdominal ivc. a second dose of 4μ (0.2 ml, male-160 mμ/g, female-200 mμ/g) was given by intraperitoneal injection after closure of the abdomen.

### Ultrasound

Ultra-high frequency ultrasound (VEVO 2100; Visual Sonics) was performed on mice at days 3, 7, and 10 following scaffold implantation to assess patency, as determined by flow through the lumen of the scaffold. prior to imaging, mice were induced with 4% isoflurane (Baxter) in 99% oxygen at 1 1/min. during imaging, isoflurane was maintained between 1.5 and 2%. The abdominal hair of the mice was shaved and ultrasound gel (AQUASONIC® clear, Fairfield, NJ, USA) was applied to the abdomen. long-axis images were acquired in b-mode to identify the scaffold, then color doppler was used to assess flow, and pulse-wave color doppler was performed to confirm that flow was non-pulsatile.

### Explanting TEVGs

Mice were euthanized with an overdose cocktail containing 200 mg/kg ketamine and 20 mg/kg xylazine. a midline laparotomy incision was created, then the ivc and aorta were bluntly dissected to isolate the TEVG. Next, a thoracotomy was performed and the right atrium was transected. An 18 g needle was then inserted into the left ventricle and the mouse vasculature was perfused with 10 ml of heparinized saline to flush the circulatory system of blood followed by 10 ml of 10% neutral buffered formalin to achieve perfusion fixation. TEVGs were removed by transecting the IVC at the anastomoses and the cranial end of the TEVG was labeled using a suture so that orientation could be preserved during histological processing.

### Histology

Excised TEVGs were fixed in 10% neutral buffered formalin overnight, then serially dehydrated through graded alcohol and xylene before paraffin embedding. samples were sectioned at a thickness of 4 μm from the middle of the TEVG. Hematoxylin and eosin (H&E) stained sections were used for quantifying histomorphometry. Collagen was identified by its characteristic birefringence following picrosirius red fast green staining and imaging under polarized light. Endothelial cells were identified by immunohistochemistry using anti-CD31 antibody (1:250, ABCAM, ab28364). Primary antibody binding was detected with biotinylated goat anti-rabbit IgG (1:1500, Vector Laboratories) followed by Vectastain Elite ABC-HRP reagent (VECTOR PK-7100) and subsequent chromogenic development with 3,3-diaminobenzidine (vector). Blood and lymphatic vessels were identified and distinguished using dual immunofluorescence. as rabbit was the host species for both primary antibodies, serial staining was first performed with anti-LYVE-1 (1:1000, ABCAM AB14917) and ALEXA FLUOR 647 conjugated goat anti-rabbit antibody (Invitrogen, A-32733) followed by anti-VWF (1:1000, DAKO a0082) and ALEXA FLUOR 488 conjugated goat anti-rabbit antibody (Invitrogen, A-11008). Images were obtained with a ZEISS AXIO Imager.A2 microscope (Carl Zeiss, Oberkochen, Germany).

### Image Analysis

Image processing and quantification was performed in IMAGEJ (NIH, Bethesda, MD). The scaffold and luminal neotissue regions were identified by manually circumscribing the outer and inner surfaces of the scaffold as well as the luminal surface of the TEVG. Luminal neotissue is reported as a fractional area representing the measured cross-sectional area of luminal tissue divided by the luminal area of a scaffold, assuming an inner diameter of 0.91 mm. occluded scaffolds were assigned a luminal neotissue area of 100%. Collagen was quantified as a fractional area of staining. blood and lymphatic vessels were identified manually; vessel lumens were circumscribed allowing for measurement of luminal area, perimeter, and a count of vessels for each histological section.

### Statistical Analysis

Statistical analysis was performed using GRAPHPAD PRISM 9. Patency outcomes were compared using Fisher's Exact Test with Bonferroni correction for multiple comparisons. Luminal neotissue (% area), collagen (% area), and vessel metrics were tested for normality using the Shapiro-Wilk test, except for intramural blood and lymphatic vessel lumen area which was tested for lognormality. Since many groups failed the test for normality, significant differences between groups were identified using the nonparametric Kruskal-Wallis and Dunn's multiple comparison tests or the Mann-Whitney test when only two of the four groups contained multiple replicates. a p value<0.05 was considered statistically significant. data are expressed as mean±sem.

### Results

Unseeded TEVGs were implanted into P2Y12 KO mice which carry a global deletion of the P2Y12 receptor that binds to ADP and potentiates platelet activation. To determine whether pharmacological inhibition of P2Y12 would prevent stenosis, PRASUGREL was administered to WT mice two hours prior to TEVG implantation (n=14).

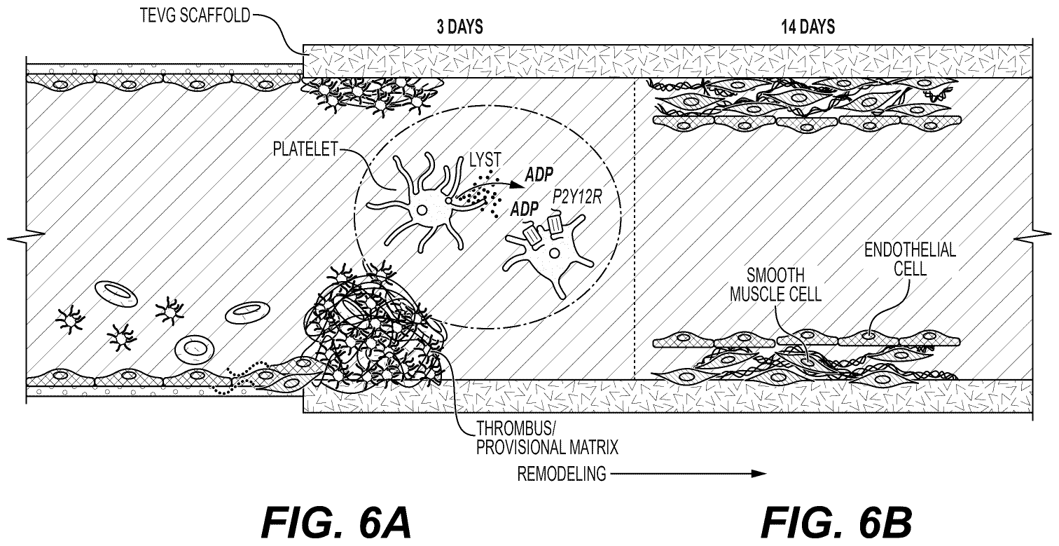

Ultrasound and Micro-CT analysis were used to analyze the TEVGs. Both P2Y12 KO and inhibition of P2Y12 using Prasugrel were effective at improving TEVG outcomes. FIGS. 7A and 7B are graphs of minimum luminal diameter (mm) for wild type, P2Y12 knock out animals, and prasugrel-treated wild-type animals, showing that the luminal diameter is significantly greater in both the knockout animals and the Prasugrel-treated animals.

The pharmacological approach uses PRASUGREL to inhibit purinergic signaling through the P2Y12 receptor in normal animals. The genetic approach uses genetic deletion of the P2Y12 receptor to inhibit ADP-mediated platelet aggregation. Wild type and P2Y12 knock out animals were compared to PRASUGREL-treated animals. The results showed that the luminal diameter is significantly greater in both the knockout animals and the PRASUGREL-treated animals (i.e., there is less stenosis).

Both P2Y12 KO and inhibition of P2Y12 using PRASUGREL were effective at improving TEVG outcomes. The key receptor is the P2Y12 receptor. This is demonstrated by the results demonstrating that PRASUGREL effectively prevents TEVG stenosis/occlusion by inhibiting ADP-mediated platelet signaling through the P2Y12 receptor and the results with the P2Y12 KO mice. The luminal diameter significantly improved in the P2Y12 KO group (1.0±0.10 mm) and the group had an overall 100% patency rate (n=24, 12/sex) (p<0.0001 Fisher's Exact Test). P2Y12 KO platelets could not aggregate in response to ADP and also had slightly decreased aggregation when exposed to thrombin and collagen. P2Y12 KO platelets still adhered to the scaffold and developed a provisional matrix that appeared to remodel into collagen-rich neotissue by 14 days.

The data show PRASUGREL, an irreversible P2Y12 antagonist, prevents TEVG stenosis without the requirement for cell seeding.

The luminal diameter significantly improved in the P2Y12 KO group (1.0±0.10 mm) and the group had an overall 100% patency rate (n=24, 12/sex) (p<0.0001 Fisher's Exact Test). P2Y12 KO platelets could not aggregate in response to ADP, and also had slightly decreased aggregation when exposed to thrombin and collagen. P2Y12 KO platelets still adhered to the scaffold and developed a provisional matrix that appeared to remodel into collagen-rich neotissue by 14 days. Ultrasound and Micro-CT analysis revealed that TEVGs were widely patent at day 3 and 100% of TEVGs remained widely patent by day 14 (1.0±0.12 mm) (p<0.0001|Fisher's Exact Test).

Similar to patent WT TEVGs, PRASUGREL-treated mice exhibited neotissue formation characterized by an endothelial cell-lined lumen (CD31) surrounded by subendothelial layers of smooth muscle cells (MyH11), resembling native veins. This finding demonstrates that PRASUGREL treatment did not affect luminal endothelialization at 14 days.

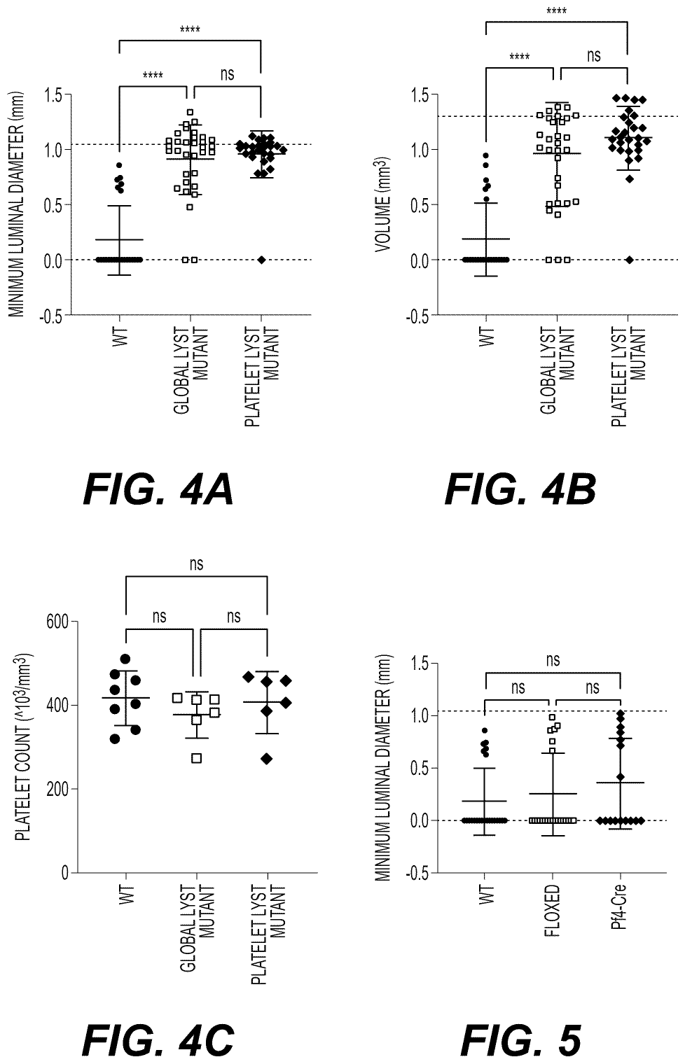

ADP released from dense granules supports proper thrombus formation by reinforcing platelet activation and aggregation through the P2Y12 receptor. See FIGS. 6A, 6B. However, ADP interactions with P2Y12 are not necessarily required for platelet adherence to blood proteins adsorbed to the luminal TEVG surface. Given that ADP potentiates sustained platelet aggregation via the P2Y12 receptor, inhibiting this pathway could reduce stenosis by interfering with thrombus stabilization. The studies in P2Y12 knockout and PRASUGREL-treated mice revealed that neither group developed stenosis post-implantation. Importantly, PRASUGREL effectively inhibited stenosis while preserving vascular neotissue formation at 14-days. This finding shows that inhibiting ADP-mediated platelet signaling could prevent aberrant remodeling without hindering vascular neotissue formation.

To disrupt the acute platelet signaling that initiates stenosis, the therapeutic must be administered prior to surgery to ensure that all platelets were exposed to the agent. These findings highlight the importance of timing and targeted modulation of platelet function to balance the need for platelet-mediated healing while preventing excessive neotissue formation.

Currently, in the clinical setting, TEVGs are implanted as scaffolds seeded with autologous bone marrow cells. Initially, bone marrow cell seeding was implemented as a strategy to accelerate endothelization promoting early healing. However, it was found that cell seeding was not required for neovessel formation but rather reduced stenosis in a dose-dependent manner and reduced macrophage infiltration into the scaffold. In clinical trials, a number of patients implanted with seeded scaffolds still developed stenosis. Further investigations revealed that seeded bone marrow cells had antiplatelet effects, suggesting that the seeded cells may reduce stenosis by disrupting the hemostatic response, potentially preventing the accumulation of ADP in the TEVG microenvironment and altering the subsequent immune response. As demonstrated in this study, antiplatelet agents like PRASUGREL help balance the acute foreign body response, which abrogates the need for bone marrow cell seeding, thereby simplifying production and broadening access to TEVGs for cardiovascular treatments, especially for facilities without specialized cell-seeding capabilities.

ADP-mediated platelet signaling appears to be the initial signal or a necessary cofactor that leads to stenosis; however, wound healing and remodeling of the provisional matrix into neotissue still requires the function of other cells, indicating that immune cells other than platelets can also influence TEVG outcomes. Since the neutrophil and macrophage LYST mutants did not affect the incidence of stenosis, their potential roles in stenosis and resolution of stenosis appear to either be independent of LYST function or act downstream of the platelet signaling. Macrophage depletion prohibits neotissue formation, yet in macrophage depleted models, platelet adherence and aggregation can still occur. Considering that LYST mutant macrophage models do not affect stenosis, it may be that while provisional matrix formation is necessary to initiate neotissue formation, macrophage function is essential for remodeling the initial thrombus into functional neotissue. Upon activation, platelets release many proteins and small molecules that act as inflammatory mediators and chemoattractants. ADP itself not only amplifies platelet aggregation but has also been shown to accelerate wound healing and fibroblast proliferation.

TEVGs serve as a model for understanding autologous tissue regeneration and the interplay between the immune and biomechanical factors that drive remodeling. In situ tissue engineering capitalizes on the body's own healing mechanisms to drive regeneration and remodeling at the site of implantation while allowing for external influence through various medical and/or procedural interventions. Furthermore, while TEVG is used for cardiovascular applications, this in situ approach is broadly applicable to other tissue engineering strategies including bone, soft tissue, and valvular structures where scaffolds leverage immune and biomechanical stimuli for effective regeneration. These models demonstrate that immune cells are not just responders but active regulators of tissue remodeling. Strategically controlling the immune microenvironment at the site of implantation can enhance the quality of tissue regeneration across a range of clinical contexts.

Modulation of platelet function therapeutic strategy targets dense granule signaling to balance TEVG narrowing with neotissue formation. ADP released from dense granules supports proper thrombus formation by reinforcing platelet activation and aggregation through the P2Y12 receptor, but its interaction with P2Y12 is not essential for platelet adherence to blood proteins adsorbed to the luminal TEVG surface. Recognizing that ADP potentiates sustained platelet aggregation via the P2Y12 receptor, inhibiting this pathway has now been shown to reduce stenosis by disrupting thrombus stabilization in wild type animals, mimicking the partial platelet dysfunction observed in Lyst mutants. P2Y12 is a common target of antiplatelet therapies, such as Prasugrel, an irreversible P2Y12 receptor antagonist. Neither P2Y12 knockout nor Prasugrel-treated mice developed TEVG stenosis post. Importantly, Prasugrel effectively inhibited stenosis while preserving vascular neotissue formation at 14-days, indicating that inhibiting ADP-mediated platelet signaling prevents aberrant remodeling without hindering vascular neotissue formation.

In conclusion, this study demonstrates that platelets are the initiators of LYST-mediated TEVG stenosis and that stenosis results from uncontrolled platelet aggregation ensuing from the acute foreign body response to the scaffold. The data show that PRASUGREL, an irreversible P2Y12 antagonist, can be used to prevent TEVG stenosis without the requirement for cell seeding.

Previous findings showed that antiplatelet agents, such as clopidogrel administered postoperatively with aspirin, partially reduce stenosis. While aspirin alone is ineffective, clopidogrel, like Prasugrel, is an irreversible P2Y12 antagonist that should also prevent the uncontrolled platelet aggregation that leads to aberrant remodeling. Variations in timing may explain the differences in effectiveness: that administering postoperatively will prove ineffective, supported by Prasugrel demonstrating superior outcomes when given before implantation. To disrupt the acute platelet signaling that initiates stenosis, the therapeutic must be administered prior to surgery to ensure that provisional matrix formation is necessary to initiate neotissue formation, but macrophages are essential for remodeling the initial thrombus into functional neotissue. Upon activation, platelets release many proteins and small molecules that act as inflammatory mediators and chemoattractants. ADP itself not only amplifies platelet aggregation but has also been shown to accelerate wound healing and fibroblast proliferation. While there are many molecules involved in wound healing, transforming growth factor-beta (TGF-β) and platelet derived growth factor (PDGF) have well documented roles in vascular repair. TGF-β, released by both platelets and other immune cells, could enhance platelet ADP reactivity and influence immune responses during and downstream of provisional matrix formation. Consistent with this notion, TGF-β receptor 1 inhibition reduced murine TEVG stenosis rates. The treatment was originally pursued based on the hypothesis that intimal hyperplasia contributed to TEVG stenosis, but during this study, the potential for TGF-β to influence platelet reactivity was not considered. TGF-β could also be involved in the crosstalk of platelets with subsequent infiltrating cells, potentially modulating processes such as the transition of monocytes to macrophages, the proliferation and migration of both smooth muscle and endothelial cells, or extracellular matrix remodeling. Similarly, PDGF contributed to vascular neotissue formation by regulating smooth muscle cell proliferation, and extracellular matrix deposition. The transient development of a provisional matrix on the TEVG appears to be the first step in neotissue development. Then, this provisional matrix acts as significant source of cytokines and growth factors, such as TGF-β and PDGF, that facilitate further immune interactions and neovessel formation. The connection between P2Y12 and Lyst appears to be significant, albeit complex. Both P2Y12 and Lyst play critical roles in platelet function and thrombosis, though they act through distinct mechanisms. P2Y12 is involved in ADP-mediated signaling pathways, promoting platelet activation and aggregation, while Lyst regulates lysosomal trafficking and dense granule secretion, which affects the release of ADP and other bioactive molecules essential for hemostasis and thrombosis. Despite differences in mechanism, both pathways converge in modulating ADP-mediated platelet function. This overlap indicates that Lyst mutations impair platelet aggregation by indirectly limiting ADP availability, similar to how P2Y12 inhibition prevents ADP-mediated platelet function by limiting receptor activation. Regardless, both P2Y12 inhibition and Lyst mutations in preventing TEVG stenosis suggests effective therapeutic approaches.

The results showed that the luminal diameter is significantly greater in both the knockout animals and the PRASUGREL-treated animals (i.e., there is less stenosis). Therefore, both P2Y12 KO and inhibition of P2Y12 using PRASUGREL were effective at improving TEVG outcomes. The data show PRASUGREL, an irreversible P2Y12 antagonist, prevents TEVG stenosis without the requirement for cell seeding.

Modulation of platelet function is the basis of the therapeutic strategy: targeting dense granule signaling to balance TEVG narrowing with neotissue formation. ADP released from dense granules supports proper thrombus formation by reinforcing platelet activation and aggregation through the P2Y12 receptor, but its interaction with P2Y12 is not essential for platelet adherence to blood proteins adsorbed to the luminal TEVG surface. Recognizing that ADP potentiates sustained platelet aggregation via the P2Y12 receptor, it was hypothesized that inhibiting this pathway could reduce stenosis by disrupting thrombus stabilization, mimicking the partial platelet dysfunction observed in Lyst mutants. P2Y12 is a common target of antiplatelet therapies, such as Prasugrel, an irreversible P2Y12 receptor antagonist. Neither P2Y12 knockout nor Prasugrel-treated mice developed TEVG stenosis post. Importantly, Prasugrel effectively inhibited stenosis while preserving vascular neotissue formation at 14-days, suggesting that inhibiting ADP-mediated platelet signaling prevents aberrant remodeling without hindering vascular neotissue formation.

This study builds on previous findings showing that antiplatelet agents, such as clopidogrel administered postoperatively with aspirin, partially reduced stenosis. While aspirin alone was ineffective, clopidogrel, like Prasugrel, is an irreversible P2Y12 antagonist that should also prevent the uncontrolled platelet aggregation that leads to aberrant remodeling. Variations in timing may explain the differences in effectiveness: that administering postoperatively will prove ineffective, supported by Prasugrel demonstrating superior outcomes when given before implantation.

To disrupt the acute platelet signaling that initiates stenosis, the therapeutic must be administered prior to surgery to ensure that provisional matrix formation is necessary to initiate neotissue formation, but macrophages are essential for remodeling the initial thrombus into functional neotissue. Both P2Y12 and Lyst play critical roles in platelet function and thrombosis, though they act through distinct mechanisms. P2Y12 is involved in ADP-mediated signaling pathways, promoting platelet activation and aggregation, while Lyst regulates lysosomal trafficking and dense granule secretion, which affects the release of ADP and other bioactive molecules essential for hemostasis and thrombosis. Despite differences in mechanism, both pathways converge in modulating ADP-mediated platelet function.

Inhibiting prolonged aggregation with Prasugrel is a successful strategy for optimizing neotissue formation and improving overall TEVG performance. Lyst mutations prevent stenosis of small-diameter TEVGs.

### Example 3: Comparison of Short Term Low Dose Losartan, Single High Dose Losartan, Short Term High Lose Losartan, Long-Term High Dose Losartan and Short Term Losartan with Bone Marrow Cell Seeding on Macrophage Infiltration, Patency of Implanted TEVG, and Survival

### Materials and Methods

The methods described above were used in these studies.

Wild type mice were untreated (control), treated with short term low dose losartan (two weeks 0.6 g/L), long-term high dose (6 months, 0.6 g/L), and short term losartan with bone marrow cell seeding. N=8/group.

Absolute numbers of Ly6C/F480⁺ macrophages was measured.

MNC infiltration and patency of implanted TEVGs was evaluated at two weeks and six months.

Survival at six months and one year was measured. N=25/group. *P<0.05; **P<0.01.

### Results

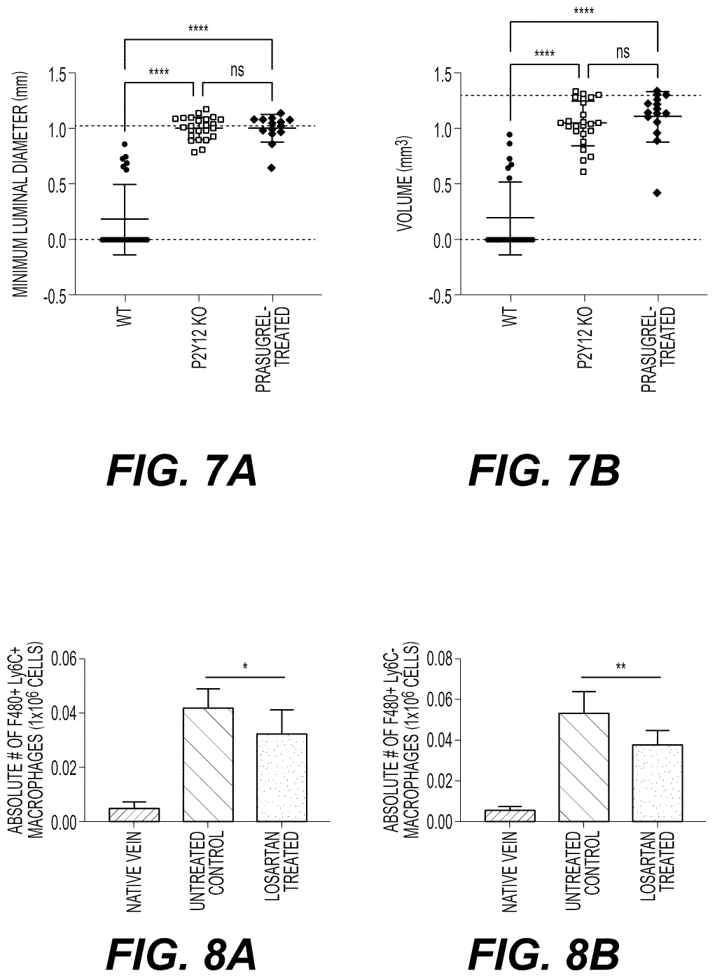

Results are shown in FIGS. 8A-8F. Short-term losartan treatment was slightly more effective in recruiting macrophages (FIGS. 8A and 8B) and in increasing patency (approximately 80%) as compared to long-term losartan treatment (approximately 65%). Compare FIGS. 8C, 8D and FIG. 8E.

Percent survival over 24 weeks was highest for short term losartan treatment combined with bone marrow cell seeding (100%). Short term losartan without cell seeding showed slightly lower survival at 24 weeks, (80%), with long term high dose losartan showing only 70% survival at 24 weeks.

### Example 4: Administration of Prasugrel with Losartan

Implantation of a foreign body (i.e., a TEVG scaffold, PTFE graft, etc.) induces a foreign body reaction characterized by a time dependent infiltration of cells into the foreign body beginning with platelets, then lymphocytes, followed by monocytes and macrophages. Prasugrel and Losartan block this pathway at different points through a different mechanism of action (prasugrel blocks the P2Y12 pathway which prevent platelet aggregation on the surface of the foreign body, while losartan blocks angiotensin II receptor 1 which prevents release of monocytes form the spleen which serves as a reservoir for the monocytes/macrophages for the foreign body reaction. Wild-type mice were implanted with TEVGs and the efficacy of treating with both Prasugrel and Losartan to prevent stenosis was examined.

### Materials and Methods

Methods and materials were as described above.

8-week-old female mice were implanted with TEVGs.

In the first group prasugrel and losartan were administered in combination with bone marrow cell seeding of the TEVG prior to implantation.

In the second group, mice were treated with prasugrel as described in example 2, with no cell seeding.

The third group were control wild-type animals implanted with unseeded TEVG and no drugs.

The study was concluded when mice were 32 weeks old.

### Results

It was theorized that combining drugs with different mechanisms of action would be more effective, even though they act on different cell types and block the pathway at a different point through a different mechanism of action.

Losartan was used to reduce TEVG stenosis by inhibiting the recruitment of monocytes from the spleen. Losartan is an antihypertensive drug that reversibly blocks angiotensin II Type 1 (AT1) receptors (AT1R). By blocking the AT1R on vascular smooth muscle cells, losartan induces vasorelaxation leading to a decrease in blood pressure. Losartan blocks angiotensin II receptor 1 which prevents release of monocytes from the spleen, which serves as a reservoir for the monocytes/macrophages for the foreign body reaction.

Prasugrel is an anti-platelet medication and a prodrug. The active metabolite of prasugrel irreversibly binds the P2Y12 receptor for adenosine diphosphate (ADP). Inhibition of platelet P2Y12 signaling inhibits platelet activation and aggregation and the stabilization of platelet aggregates as well as promotes disaggregation leading to smaller thrombus size.

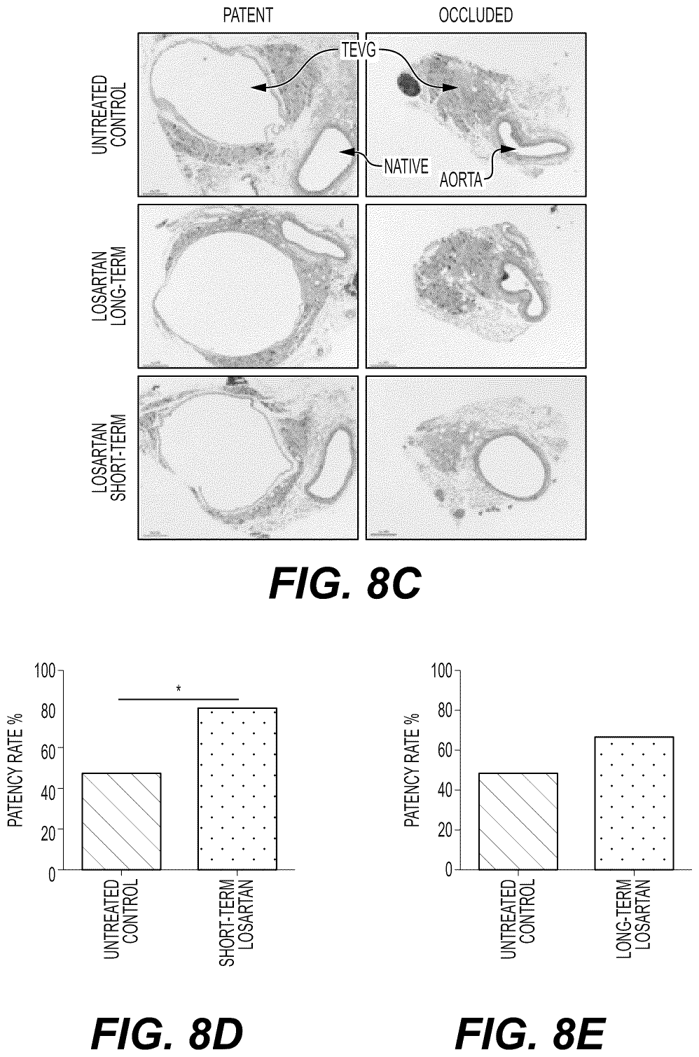

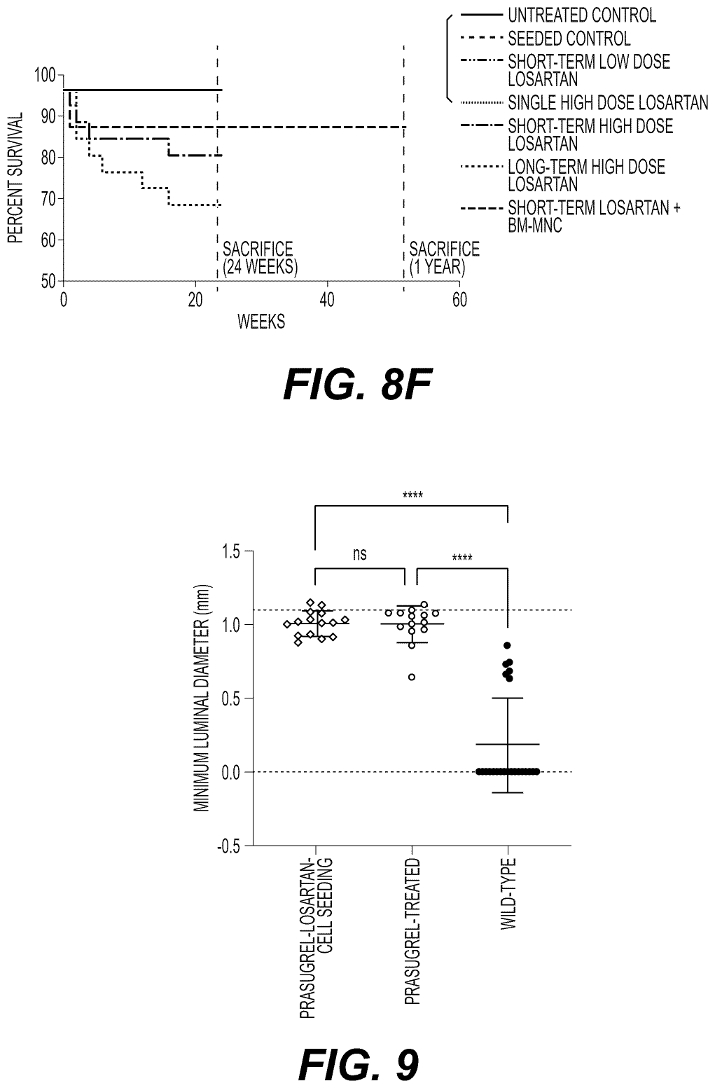

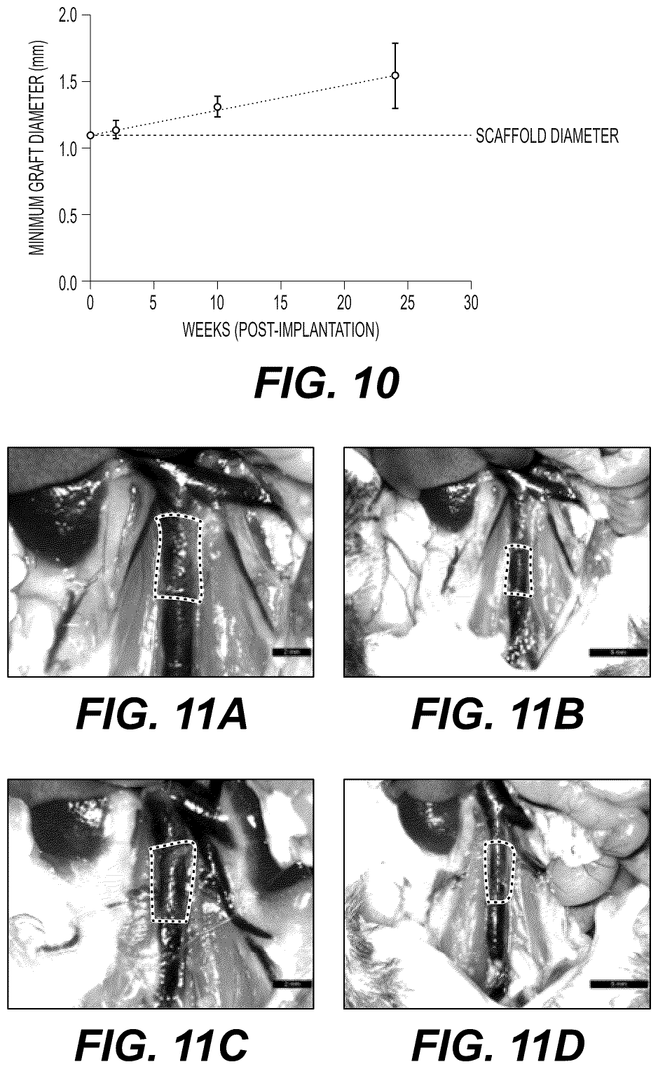

The mice were 8 weeks old when they were implanted with TEVGs and were 32 weeks old at their endpoint. FIG. 9 shows minimum TEVG diameter over time in the prasugrel-losartan-cell seeded group (n=14) compared to prasugrel treated and wild type animals. FIG. 10 is a graph of minimum luminal diameter (mm) over time (weeks). The starting scaffold diameter is indicated with the dotted line. At two weeks, the prasugrel-losartan-cell seeding treatment resulted in 100% group patency. The treatment was not significantly different than prasugrel-treatment with unseeded TEVGs alone, which also had group patency of 100%, showing that using prasugrel obviates the need for cell seeding. The WT mice have a group patency of ˜25%. FIGS. 11A-11D is a comparison of patent (A, C on left) versus occluded (B, D on right) vessels following treatment.

Implantation of a foreign body (i.e., a TEVG scaffold, PTFE graft, etc.) induces a foreign body reaction characterized by a time dependent infiltration of cells into the foreign body beginning with platelets, then lymphocytes, followed by monocytes and macrophages. Each drug works blocks this pathway at a different point through a different mechanism of action (prasugrel block the P2Y12 pathway which prevent platelet aggregation on the surface of the foreign body, and losartan blocks angiotensin II receptor 1 which prevents release of monocytes form the spleen which serves as a reservoir for the monocytes/macrophages for the foreign body reaction).

The combination of prasugrel and losartan completely eliminate stenosis without bleeding complications in the TEVG mouse model. In contrast, losartan alone or combined with cell seeding reduced the incidence of stenosis but did not eliminate it. The potential risk to this strategy included an increase in bleeding complications or excessive blockage of the foreign body reaction which could block the vascular neotissue formation. The data demonstrates losartan, prasugrel, and cell seeding completely blocked stenosis without bleeding complications or adversely effecting neovessel formation.

1. A dosage unit for reducing thrombosis or stenosis of tissue engineered grafts, vascular grafts, stents, patches or valves comprising an effective amount of an inhibitor of platelet aggregation selected from the group consisting of inhibitors of ADP mediated platelet signaling through the P2Y1, P2X1 or P2Y12 receptor on the surface of platelets, angiotensin II receptor blocker (ARB) inhibitors, and angiotensin-converting enzyme (ACE) inhibitors to reduce thrombosis or stenosis of the graft following implantation, angioplasty or anastomosis in the vasculature of an individual in need thereof, wherein the dosage is provided in an amount covering the day prior to the implantation, angioplasty or anastomosis, and for a period thereafter until the endothelial surface of the vasculature disrupted by the implantation, angioplasty or anastomosis is restored.

2. Tissue engineered grafts, vascular grafts, stents, patches or valves comprising an effective amount of an inhibitor of platelet aggregation selected from the group consisting of inhibitors of ADP mediated platelet signaling through the P2Y1, P2X1 or P2Y12 receptor on the surface of platelets, angiotensin II receptor blocker (ARB) inhibitors, and angiotensin-converting enzyme (ACE) inhibitors to reduce thrombosis or stenosis of the tissue engineered grafts, vascular grafts, stents, patches or valves following implantation, angioplasty or anastomosis in the vasculature of an individual in need thereof, wherein the dosage unit is sufficient to provide an effective amount covering the day prior to the implantation, angioplasty or anastomosis, and for a period thereafter until the endothelial surface of the vasculature disrupted by the implantation, angioplasty or anastomosis is restored.

3. The tissue engineered grafts, vascular grafts, stents, patches or valves of claim 2 comprising an effective amount of an inhibitor of platelet aggregation selected from the group consisting of inhibitors of ADP mediated platelet signaling through the P2Y1, P2X1 or P2Y12 receptor on the surface of platelets, angiotensin II receptor blocker (ARB) inhibitors, and angiotensin-converting enzyme (ACE) inhibitors in an extended release formulation to reduce thrombosis or stenosis of the tissue engineered grafts, vascular grafts, stents, patches or valves following implantation, angioplasty or anastomosis in the vasculature of an individual in need thereof.

4. The tissue engineered grafts, vascular grafts, stents, patches or valves of claim 2 wherein the inhibitor is released in an effective amount to prevent platelet aggregation over a period of 48-72 hours following implantation, angioplasty or anastomosis of the tissue engineered grafts, vascular grafts, stents, patches or valves.

5. The tissue engineered grafts, vascular grafts, stents, patches or valves of claim 2 wherein the inhibitor is selected from the group consisting of Prasugrel, clopidogrel, ticlopidine, and ticagrelor.

6. The dosage unit of claim 1 wherein the inhibitor is Prasugrel.

7. The tissue engineered grafts, vascular grafts, stents, patches or valves of claim 2 comprising an ARB inhibitor, an ACE inhibitor, or both an ARB inhibitor and an ACE inhibitor.

8. The tissue engineered grafts, vascular grafts, stents, patches or valves of claim 2 comprising Losartan.

9. The dosage unit of claim 1 comprising Prausugrel in a daily dosage of about 1 mg/kg.

10. The dosage unit of claim 1 comprising losartan in an amount providing about 0.1 mg losartan/kg/day.

11. The dosage unit of claim 1 comprising an oral dosage unit package, with dosages separated by time of administration from prior to the surgery to the conclusion of the treatment.

12. The dosage unit package of claim 11 comprising a two-week course of losartan and Prasugrel, providing an initial dose of Prasugrel for adults weighing more than 60 kg, of 60 mg orally once followed by a 10 mg maintenance dose, and a dose of 5-10 mg losartan is administered orally once or a dose of 0.1 mg/kg/day.

13. A method for reducing thrombosis or stenosis of tissue engineered grafts, vascular grafts, stents, patches or valves comprising administering to an individual in need thereof an effective amount of an inhibitor of platelet aggregation selected from the group consisting of inhibitors of ADP mediated platelet signaling through the P2Y1, P2X1 or P2Y12 receptor on the surface of platelets, angiotensin II receptor blocker (ARB) inhibitors, and angiotensin-converting enzyme (ACE) inhibitors to reduce thrombosis or stenosis of the graft following implantation, angioplasty or anastomosis in the vasculature of an individual in need thereof.

14. The method of claim 13, wherein the inhibitor is provided in an amount covering the day prior to the implantation, angioplasty or anastomosis, and for a period thereafter until the endothelial surface of the vasculature disrupted by the implantation, angioplasty or anastomosis is restored.

15. The method of claim 13 wherein the inhibitor is selected from the group consisting of Prasugrel, losartan and combinations thereof.

16. The method of reducing thrombosis or stenosis of vascular grafts of claim 13 comprising administering prior to or at the time of implantation of the vascular graft, an effective amount of an inhibitor of ADP-mediated signaling or the purinergic receptors P2Y1 and P2X1 on the surface of platelets to reduce thrombosis or stenosis of the graft following implantation in an individual in need thereof.

17. The method of claim 16 wherein the inhibitor is administered within approximately 2 hours before surgery for implantation of the vascular graft in an effective amount to reduce thrombosis or stenosis of vascular grafts.

18. The method of claim 16 where the inhibitor is incorporated into or onto the vascular graft to release following implantation.

19. The method of claim 16 wherein the inhibitor is PRASUGREL.

20. The method of claim 19 wherein the PRASUGREL is administered in a dosage of about is 1 mg/kg.

21. The method of claim 13 further comprising administering to the patient an angiotensin II inhibitor such as losartan, preferably in a dose such as 0.1 mg/kg/day.
